(19) United States
(12) Patent Application Publication
(10) Pub. No.: US 2022/0310246 A1
(43) Pub. Date: Sep. 29, 2022

(54) SYSTEMS AND METHODS FOR QUANTITATIVE ASSESSMENT OF A HEALTH CONDITION

(71) Applicants: Thomas Blomseth Christiansen, Copenhagen (DK); Danmarks Tekniske Universitet, Kgs. Lyngby (DK)

(72) Inventors: Jakob Eg Larsen, Vaerlose (DK); Thomas Blomseth Christiansen, Copenhagen (DK)

(73) Assignee: Danmarks Tekniske Universitet, Kgs. Lyngby (DK)

(21) Appl. No.: 17/211,589

(22) Filed: Mar. 24, 2021

(51) Int. Cl. G16H 40/67; G16H 10/60; G16H 50/70; G16H 70/20; G16H 10/20; G16H 50/20; G16H 20/70; G16H 50/30; H04L 12/58; A61B 5/0205; A61B 5/16

(52) U.S. Cl. CPC G16H 40/67; G16H 10/60; G16H 50/70; G16H 70/20; G16H 10/20; G16H 50/20; G16H 20/70; G16H 50/30; H04L 51/02; A61B 5/02055; A61B 5/16; A61B 5/02405

(57) ABSTRACT

Systems and methods for assessment of one or more suspected conditions of an individual that include receiving collaborative identification data pertaining to features of a suspected condition and subject-specific phenomena of presentation, selecting target phenomena, receiving quantitative hypotheses for the target phenomena, defining an observation protocol comprising instructions for how the individual is to self-monitor and register observations by interaction with a portable data acquisition system, upon each occurrence of the target phenomena, directing the individual to provide the stored data upon observation of a target phenomenon during observation periods, transmitting the stored data from the portable data acquisition system to the analysis component, directing the individual to recall each observation period, completing one or more quantitative comparisons between any of the stored data, the quantitative hypotheses, and the recollection, inferring the suspected conditions, and determining a continued progress of the assessment.

### FIELD

The present disclosure relates to methods and devices for assessment and treatment of health-related conditions. A method allows for quantitative assessment of one or more suspected conditions of an individual comprising an observation period wherein the individual is instructed to register observations of occurrences of one or more target phenomena, related to said suspected condition, by interaction with a portable data acquisition system.

### BACKGROUND

Assessment of health-related conditions, and in particular mental-health related conditions, has for long been based on a linear and static course of action. Conventional assessments typically have to rely on information provided by the individual's description of the symptoms and occurrences. Such information is prone to response bias. In addition, the assessments may be guided by the limited information provided by questionnaires, such as the PCL-5, which is a 20-item self-report measure that assesses 20 symptoms of PTSD, as defined in DSM-5.

A challenge with therapeutic work within this general frame is the quality of the information provided to the mental health counselor, e.g. a psychotherapist or a therapist. As outlined above, there are several negative aspects with the present methodologies that may lead to a loss in crucial information during the assessment, which could have been decisive in disentangling different symptoms and their causes. Thereby, the mental health counselor may not be equipped with adequate information for making well-informed conclusions about the condition of the individual. Accordingly, there exists a need for systems that can obtain data from a subject in substantially real time during a monitoring period and subsequently provide the obtained data and associated recommendations for mental health counselors to use when meeting with the subject after the monitoring period.

### SUMMARY

In a first aspect, a method for assessment of one or more suspected conditions of an individual includes receiving, by an analysis component, collaborative identification data pertaining to one or more features of a suspected condition and one or more subject-specific phenomena of presentation. The method further includes selecting, by the analysis component, one or more target phenomena from the one or more subject-specific phenomena of presentation. The method further includes receiving, by the analysis component, one or more quantitative hypotheses for the one or more target phenomena. The method further includes defining, by the analysis component, an observation protocol comprising instructions for how the individual is to self-monitor and register observations in the form of stored data, by interaction with a portable data acquisition system communicatively coupled to the analysis component, upon each occurrence of the target phenomena is observed by the individual. The method further includes directing, by the portable data acquisition system, the individual to provide the stored data to the portable data acquisition system in accordance with the observation protocol upon observation of a target phenomenon of the one or more target phenomena during one or more observation periods. The method further includes transmitting the stored data from the portable data acquisition system to the analysis component. The method further includes directing the individual to recall each observation period. The method further includes completing, by the analysis component, one or more quantitative comparisons between any of the stored data, the one or more quantitative hypotheses, and the recollection. The method further includes inferring, based on the quantitative comparison, the one or more suspected conditions of the individual. The method further includes determining, based on the inferred one or more suspected conditions of the individual, a continued progress of the assessment of the individual.

In another aspect, a system for assessment of one or more suspected conditions of an individual, includes an assessment device, a portable data acquisition system communicatively coupled to the assessment device, and an external device coupled to the assessment device. The assessment device is configured to receive collaborative identification data pertaining to one or more features of a suspected condition and one or more subject-specific phenomena of presentation, receive one or more quantitative hypotheses for the one or more target phenomena, and define an observation protocol comprising instructions for how the individual is to self-monitor and register observations. The portable data acquisition system is configured to receive the registered observations from the individual in accordance with the observation protocol upon observation of a target phenomenon of the one or more target phenomena during one or more observation periods. The assessment device is further configured to obtain the data from the portable data acquisition system, direct the individual to recall each observation period, complete one or more quantitative comparisons between any of the stored data, the quantitative hypotheses, and the recollection, infer, based on the quantitative comparison, the one or more suspected conditions of the individual, and determine, based on the inferred one or more conditions of the individual, a continued progress of the assessment of the individual. The external device receives a data file from the assessment device, the data file containing the continued progress of the assessment of the individual and presents the data file to a user.

It is to be understood that both the foregoing general description and the following detailed description describe various embodiments and are intended to provide an overview or framework for understanding the nature and character of the subject matter claimed and described herein. The accompanying drawings are provided to facilitate a further understanding of the various embodiments, and are incorporated into and constitute a part of this specification. The drawings illustrate the various embodiments described herein, and together with the description serve to explain the principles and operations of the subject matter claimed and described herein.

### BRIEF DESCRIPTION OF THE DRAWINGS

FIG. 1 depicts an illustrative system for quantitative assessment of a health condition according to one or more embodiments shown and described herein;

FIG. 2 depicts a portable data acquisition system according to an embodiment of the present disclosure, for registering of observations, by an individual;

FIG. 3 depicts a flow diagram outlining a method for assessment of a suspected condition of an individual, according to an embodiment of the present disclosure;

FIG. 4 depicts a flow diagram outlining an initiation session, according to a specific embodiment of the present disclosure;

FIG. 5A depicts a flow diagram outlining an observation period, according to a specific embodiment of the present disclosure;

FIG. 5B depicts an additional flow diagram outlining the observation period, according to a specific embodiment of the present disclosure;

FIG. 6A depicts a flow diagram outlining an evaluation session, according to a specific embodiment of the present disclosure; and

FIG. 6B depicts an additional flow diagram outlining the evaluation session, according to a specific embodiment of the present disclosure.

### DETAILED DESCRIPTION

The present disclosure relates to methods and systems for assessment and/or treatment of one or more suspected conditions of an individual. The methods and systems described herein could allow for the provision of accurate, precise and quantitative information related to the condition of the individual, which may be used in order to infer information about the suspected condition of the individual and the continued progress of the assessment of the individual.

A mental health condition of major concern is posttraumatic stress disorder (PTSD), which in the United States alone affects about 3.5% of adults who have PTSD in a given year, and almost a tenth of the population develop PTSD at some point in their life. PTSD is triggered by experiencing or witnessing a traumatic event. Symptoms re-experiencing the trauma (flashbacks, nightmares, or uncontrollable thoughts about the event) and avoidance of trauma-related stimuli, as well as hyperarousal, loss of temper and fear behavior even in safe environments. Getting effective, and early, treatment when PTSD symptoms develop can be critical to reduce the burden and improve function.

Existing psychotherapeutic methods commonly have an element of conversational therapy, such as the prevalent cognitive behavioral therapy (CBT). The typical course of assessment relies on three distinct steps, initial sessions (assessment, goal-setting), a series of psycho-therapeutic sessions (accompanied by homework, typically behavioral experiments and exposure training), and completion of treatment.

During the initial sessions, the therapist classifies, based on the information provided by the individual, the condition of the individual, and based on this classification instructs the individual to undergo a standard assessment that is typically provided to individuals within that specific classification.

Based on assessment information, the therapist constructs and presents a case formulation based on etiological hypotheses. Jointly, the patient and the therapist set goals for the next phase, psychotherapeutic treatment. The latter is typically carried out over an extended period of time, such as 8-16 weeks, consisting of both psychotherapeutic sessions and homework. During sessions the therapist and the individual discuss the symptoms and the therapist imparts strategies of how the individual is to handle the condition in everyday life. This may include psychoeducation as part of psychotherapeutic treatment.

At completion of the treatment the individual typically fills out questionnaires for assessing the level of symptoms. While questionnaires are commonly used, they have several limitations, for example in that they rely on the ability of the patient to accurately respond to questions. Memory can be impaired by trauma. Willingness to indicate symptoms may be influenced by the patient's relation to the therapist, or, the patient's acceptance of the disorder (e.g. shamefulness). Lastly, what the questionnaire (e.g. PCL-5) asks for may commonly be misunderstood (many questionnaires assess severity by means of symptom frequency, where a patient may respond by indicating the felt severity of a rarely occurring symptom, or, by indicating the frequent worries that a relatively infrequent symptom spurs rather than the frequency of the symptom itself).

The present disclosure overcomes past limitations by providing methods and systems for assessment and/or treatment of suspected conditions of an individual, with subjectively experienced symptoms. In a method, the individual may receive targeted therapeutic interventions based on the specifics of their individual symptom dynamics through observational data collected during everyday life in an unobtrusive way.

The method has been proven to be highly versatile and may be applied to a broad range of suspected conditions. These conditions may for example be mental health related conditions such as anxiety disorders, mood disorders, schizophrenia, substance abuse disorders, and stress-related disorders. Alternatively, or additionally, the conditions may be physiologically related health conditions with subjectively experienced symptoms such as chronic pain, gastrointestinal discomfort, and peripheral neuropathy. The method allows an examiner to gather reliable information about the suspected condition of the individual, including quantitative insights into the subject-specific symptoms of the condition.

The examiner and the individual may collaboratively identify one or more features of the suspected condition, during an initiation session. These features may comprise or consist of one or more physiological features, and/or one or more mental health related features. Furthermore, the individual and the examiner may further identify a number of subject-specific phenomena, which may be related to how the individual consciously experiences features of the suspected condition, such as bodily experienced, mentally experienced and/or environmentally experienced.

The examiner and the individual may select one or more target phenomena, among said subject-specific phenomena, wherein the target phenomena are one or more of: i. bodily experienced precursors to a mental state, such as a somatic marker or mental awareness; ii. bodily sensations, such as pain or a thought pattern; iii. awareness of mental states, such as awareness of specific cognitions, such as psychotic perceptions, ruminative thought patterns, attention towards threats, depressive cognitions; or iv. subjectively perceived phenomena, such as conscious awareness of a phenomenon, to one or more of the features of the suspected condition.

The individual may be provided with a portable data acquisition system, during an observation period, and instructed to register observations related to the target phenomena, by interaction with said portable data acquisition system. By the use of the portable data acquisition system, the individual may self-monitor and self-register observations, and thereby allow for quantitative and accurate insights related to the mental and physiological health state of the individual. The portable acquisition instrument may associate each observation with contextual and physiological information, such as point in time, location, and heart rate, in addition to information related to the observation, such as the type of target phenomenon, the duration of the interaction with the instrument, the duration of the observation, and the magnitude of the observed occurrence of the target phenomenon. Thereby, the examiner is provided with quantitative information related to the circumstances with an increased likelihood of the individual being at risk of experiencing the target phenomena, without being subject to recall or reporting bias.

The system may include one or more self-contained devices onto which the individual self-registers observations during a period of time, which includes, for example, a bodily worn component communicatively coupled to a processing and display component, such as a smartphone or the like. Typically, the individual is instructed to register observations while being exposed to common, everyday life situations, for the individual, but may further comprise subjecting the individual to challenging situations where there may be an increased likelihood that the individual will experience a target phenomenon. The user typically registers observations on the bodily worn component, the processing and display component, or on a combination of the two, as described in greater detail herein. While the system described herein incorporates both a bodily worn component and a smartphone or the like, it should be understood that the present disclosure is not limited to such. For example, in some embodiments, the system may be included wholly within a bodily worn component, such as a fitness tracker, a smart watch, or the like.

In the evaluation session, quantitative comparisons are preferably made in order to provide precise insights into the suspected condition of the individual. These quantitative comparisons may be carried out between the stored data and the quantitative hypotheses, in terms of for example the frequency of observation at different time points of the day and at different locations. Thereby, quantitative insights are provided to the examiner who can infer, based on the quantitative comparison, an updated understanding about the suspected condition of the individual.

In an embodiment of the present disclosure, the portable data acquisition system may, following registration of an observation on the bodily worn component and/or on the processing and display component, prompt the individual to perform an intervention, for example in the form of a breathing exercise or other behavioral or cognitive modifications. The portable data acquisition system may further be used to register the completion of said intervention, allowing for quantitative insights into the individual's compliance to the intervention protocol.

It may be a further preference that the bodily worn component of the portable data acquisition system is configured such that registering of observations and completion of intervention involves the least amount of effort, while said instrument simultaneously can be worn concealed or mimic commonly worn apparel. The bodily worn component of the portable data acquisition system may for example be provided as a single-button wrist-worn instrument. While the portable data acquisition system may have a simple appearance, such as a user interface, and may have only a single component for user input, said instrument may allow for registering of different types of events, such as observations, the properties of the observation, and the completion of an intervention, by event-specific interactions. Such a device may thereby allow for discrete interactions, wherein observations can be registered by the individual without other people noticing, and/or interactions that require little attention, such as visual attention, from the individual.

An examiner as used herein refers to a physical person and/or a software agent that assists individuals in alleviating health conditions and/or improving the quality of their lives. The examiner may for example be a health professional, such as a mental health professional. A typical example is a person or a software agent that specializes in the therapeutic treatment of a condition, such as an impairment, injury, disease, and/or a disorder.

A software agent, colloquially known as a chatbot, as used herein refers to a fully-automated, or semi-automated, software system, for example based on artificial intelligence techniques, that is programmed to have a dialogue with an individual. Typically, the software agent is programmed to execute therapeutic techniques. Furthermore, the software agent may provide an individual with daily chat conversations and offer self-help techniques, mood tracking, curated videos, meditation and mental exercises. The software agent may be contained within a non-transitory, processor-readable memory component integrated within one or more of the devices of the portable data acquisition system, an assessment device, and/or one or more external devices, as described in greater detail herein.

The present disclosure relates to systems and methods for assessing and potentially treating one of more suspected conditions of an individual, that comprises: i. an initiation session involving conversation between an individual and an examiner wherein a number of quantitative hypotheses about one or more target phenomena pertaining to the condition are generated; ii. an observation period wherein the individual is instructed to register one or more of said target phenomena by interaction with a portable data acquisition system, iii. an evaluation session wherein the individual recollects the observation period, the registered data is retrieved, and a number of quantitative comparisons are made between any of the stored data, the quantitative hypotheses, and the recollection, for making inferences about the suspected condition, and concluding a continued progress of the assessment of the individual's condition.

In an embodiment of the present disclosure, the method comprises an initiation session, an observation period and an evaluation session. It is a preference that the initiation session comprises communication between the examiner and the individual, and furthermore that one or more features of the suspected condition is identified. Said identification of the features of the suspected condition may be a collaborative effort, between the examiner and the individual. Furthermore, in the initiation session, one or more subject-specific phenomena of presentation are preferably identified. This step may thereby be seen as a step directed at reducing the number of target phenomena. As disclosed elsewhere herein, although counterintuitive the collection of less amount of data may be advantageous. One explanation to this may be the ability of an individual to conform to an observation protocol wherein a lower number of target phenomena are defined. These phenomena may be identified collaboratively between the examiner and the individual. In addition, one or more target phenomena are selected, preferably collaboratively between the examiner and the individual. It is a further preference that the initiation session comprises the generation of one or more quantitative hypotheses. Yet further, the initiation session in a preferred embodiment comprises the definition of an observation protocol. Said definition may be a mutual agreement of the format and content of said observation protocol. The observation protocol may comprise or consist of instructions for how the individual is to register observations by interaction with a portable data acquisition system, upon each occurrence of the target phenomenon is observed by the individual. It is a preference that each registering of an observation renders data related to said observation being stored, such as on the portable data acquisition system or at a remote server, such as a networked server, or both for synchronization of the stored data. The individual may monitor and register observations during the observation period, such as self-monitor and self-register observations respectively.

In an embodiment of the present disclosure, the observation period comprises exposing the individual to everyday life, such as its situations and activities. Said everyday life may be typical days for the individual. The everyday life may further comprise or consist of one or more periods with changed levels of symptoms, such as a time interval with worsening of symptoms. The observation period may run for a predefined time period, until certain predefined criteria are met, or until the examiner and/or the individual decides to terminate or suspend the observation protocol. During the observation period, the individual is instructed to conform to the observation protocol upon each observation of the target phenomenon. Thereby, during the observation period, the individual may self-monitor and self-register observations, such as the one or more target phenomena, resulting in stored data, and wherein said stored data comprises information related to said observations. Said data may for example comprise details regarding which target phenomena was registered, the time, the location, the duration, and the magnitude of each observation of occurrence of a target phenomenon.

Following the observation period, the next step in the assessment is typically the evaluation session. It is a preference that the evaluation session comprises communication between the examiner and the individual. Furthermore, the evaluation session preferably comprises acquisition of the stored data, such as retrieval of the stored data. Acquisition of the data may thereby for example be from the portable data acquisition system, or from a server, such as a networked server. The acquisition may lead to that said data are available during the evaluation session, for the examiner and/or the individual. The data may for example be available on a computing device, such as a computer or a tablet, in the office of the examiner. It may further be a preference, that said computing device comprises a processing unit, a power source, and a memory upon which instructions are stored that allows for the retrieval of the data. It may be a further preference that the memory comprises instructions for presentation and/or visualization of the data, or parts thereof, such as aggregate statistics computed from the data. For example by displaying the data, or parts thereof, on a monitor, or for providing a computer printout of the data or parts thereof.

In an embodiment of the present disclosure, the evaluation session comprises a recollection of the observation period or the observation period based on the registered observation of the target phenomenon, by the individual. The information provided by the recollection may be provided, such as by the examiner, to the computing device and/or the networked server. Said information may further be processed locally or remotely, and may be used to obtain recollection data and/or used for making comparisons with other data. The recollection may comprise a subjective recollection, by the individual, of the observation period and/or the time period. The recollection may comprise information related to events or activities that occurred during the observation period, and may further comprise any information that may be relevant for assessing the condition of the individual. Such information may comprise information of what the individual did, and where the individual was located, before, during and after the observations of occurrence of the target phenomenon. The recollection may for example comprise information related to where the individual was located, or what type of activities the individual was carrying out, at the instance of the observation of occurrences. The individual may for example have lunch at work, or the individual may be driving. The recollection may further comprise information of what occurred before the observation of the target phenomenon, such that it can be inferred of what event or activities that led to the observation of the target phenomenon. Furthermore, the recollection may comprise information of what occurred after the observation of the target phenomenon, such that it can be inferred of how the individual handled the situation. The recollection may comprise any information related to the observation of the target phenomenon, for example the distribution of any time period, such as the day, the week, the year, the intensities, the durations, the locations, the frequency, and the type target phenomenon. In general, the individual may recollect the observation period by providing any information that is relevant for making inferences about the suspected condition. The individual may further provide patterns of occurrences of the target phenomenon, and/or typical sets of parameters, such as one or more sets of parameters, such as time and location, for when the target phenomenon often occurred.

In a further embodiment of the present disclosure, the evaluation session comprises one or more quantitative comparisons. The quantitative comparisons preferably comprises comparisons between any of the stored data, the quantitative hypotheses, and the recollection. The quantitative comparison may thereby be a comparison between the stored data, the quantitative hypotheses, and the recollection. Alternatively, or additionally, the quantitative comparison may be a comparison between the stored data and the quantitative hypotheses. Alternatively, or additionally, the quantitative comparison may be a comparison between stored data and the recollection. Alternatively, or additionally, the quantitative comparison may be a comparison between the quantitative hypotheses and the recollection. The quantitative comparison may thereby be a comparison between any of the stored data, the quantitative hypotheses and the recollection, in any possible combination.

In yet a further embodiment of the present disclosure, the evaluation session further comprises inferring, typically based on the quantitative comparisons, an understanding of the suspected condition of the individual. The inferences may be provided by the examiner, the individual, and/or a collaborative effort. Typically, the understanding is an updated understanding, as at least parts of information related to the suspected condition was known a priori. In an embodiment of the present disclosure, the evaluation session further comprises a conclusion of the continued progress of the assessment of the individual. Said conclusion may be based on the understanding, such as the updated understanding, of the suspected condition of the individual.

### Conceptual Architecture

In a further embodiment of the present disclosure, suspected condition is a gap between the state of the individual and the desired state, given some dimensions, for example the mental state. Conditions may for example be disorders, diseases and/or syndromes, both mental health related and non-mental health related.

A condition may further comprise clinical conditions, problems, diagnoses, disorders, or other events, situations, issues, or clinical concepts that have risen to a level of concern. For clarity, it should be noted that the condition may be a combination of underlying conditions, for example the condition may comprise anxiety, depression and misuse disorder. An individual, being assessed by the present method, is commonly expected to have multiple conditions.

The multiple (suspected) conditions may be assessed individually, or may be assessed in combination, either multiple, or all conditions. In an embodiment of the present disclosure, the conditions comprises or consists of disorders or diseases, or a combination thereof, as provided by ICD-11 Version:2018, or DSM-5. In a specific embodiment of the present disclosure, the condition comprises or consists of mental disorders as provided by ICD-11 Version:2018, or DSM-5.

Thereby, mental conditions/disorders may comprise organic, including symptomatic, mental disorders, mental and behavioral disorders due to psychoactive substance use, schizophrenia, schizotypal and delusional disorders, mood affective disorders, neurotic, stress-related and somatoform disorders, behavioral syndromes associated with physiological disturbances and physical factors, disorders of adult personality and behavior, mental retardation, disorders of psychological development, behavioral and emotional disorders with onset usually occurring in childhood and adolescence, and/or any unspecified mental disorder, or a combination thereof.

Furthermore, specific examples of mental conditions/disorders may comprise or consist of any of anxiety disorder, social anxiety, post-traumatic stress disorder (PTSD), such as combat exposure PTSD, or obsessive—compulsive disorder (OCD), depression, schizophrenia, substance abuse disorder, and/or eating disorder, or a combination thereof.

In an embodiment of the present disclosure, the condition is one or more physiological health related conditions, and may further comprise a symptom including chronic pain, gastrointestinal discomfort, and peripheral neuropathy.

In an embodiment of the present disclosure, the condition is any condition comprising subjectively experienced symptoms and/or side effects that could be used to inform treatment decisions. An exemplary condition is cancer wherein the patient/individual experiences treatment induced nausea that is to be suppressed by way of dosage and timing of medication.

In a further embodiment of the present disclosure, the features of the suspected condition comprises or consists of one or more physiological symptoms, and/or one or more mental health related symptoms. Physiological symptoms may for example be pain, racing heart, shortness of breath, itching, fatigue, neuropathy, headache, migraine, chills, hot flushes, sexual arousal, muscle tension, coughing, sneezing, speech impediments and/or freeze of gait while, or a combination thereof. Similarly, mental-health related symptoms may for example be intrusive memories, avoidance, negative changes in thinking and/or mood, changes in physical and emotional reactions, flashbacks, nightmares, hypervigilance, anxiety and/or fear, or a combination thereof.

In an embodiment of the present disclosure, the features of the suspected condition comprises or consists of features, such as symptoms or typical symptoms, as provided in ICD-11 Version:2018, or in DSM-5. In a specific embodiment of the present disclosure, the features comprises or consists of mental-health related features of the suspected condition as provided in ICD-11 Version:2018, or DSM-5.

In June 2018, the ICD released its 11th version (ICD-11), which includes the diagnosis of posttraumatic stress disorder (PTSD) and complex PTSD (CPTSD; WHO, 2018), as further detailed by Cloitre et al. ICD-11 Posttraumatic Stress Disorder and Complex Posttraumatic Stress Disorder in the United States: A Population-Based Study, Journal of Traumatic Stress, 2019

In a further embodiment of the present disclosure, the subject-specific phenomena of presentation are how the individual consciously experiences features of the suspected condition, such as bodily experienced, environmentally experienced, and/or mentally experienced. The subject-specific phenomena of presentation may for example be how the condition manifests and/or presents itself specifically for that individual. Environmentally experienced may be subject-specific phenomena that the individual perceives in the environments.

In a further embodiment of the present disclosure, the subject-specific phenomena of presentation are one or more of: bodily experienced precursors to a mental state, such as a somatic marker or mental awareness; bodily sensations, such as pain or a thought pattern; or subjectively perceived phenomena, such as conscious awareness of a phenomenon, to one or more of the features of the suspected condition.

In yet a further embodiment of the present disclosure, the subject-specific phenomena of presentation are one or more of: bodily experienced precursors to a mental state, such as a somatic marker or mental awareness; environmentally experienced; mentally experienced, such as conscious awareness of a phenomenon, to one or more of the features of the suspected condition, for example: i. awareness of mental states, such as awareness of specific cognitions, such as psychotic perceptions, ruminative thought patterns, attention towards threats, depressive cognitions; or ii. bodily sensations, such as pain.

In yet a further embodiment of the present disclosure, the target phenomenon comprises or consists of any one or more of the subject-specific phenomena of presentation that: has the biggest impact on the individual's life; is bothering the individual the most; is the most directly noticeable in everyday situations of the individual; is considered to be the most relevant indicator for treatment or in relation to a diagnosis; is considered to be of greatest clinical interest and/or relevance; is believed to lead to the greatest reduction of uncertainty about the suspected condition, or a combination thereof, such as wherein different weights to the aforementioned criteria to derive a number of target phenomena; and/or is relevant for the suspected condition.

As discussed elsewhere herein, it may be counterintuitive that decreasing the number of subject-specific phenomena to only a few or maybe even a single subject-specific phenomenon would be advantageous for an assessment of a suspected condition of an individual. However, in a specific embodiment of the present disclosure the method comprises a step of reducing the observation protocol to the simplest method that is still viable for assessment of the suspected condition. Preferably the method involves selecting a few, such as one or more subject-specific phenomena, as the target phenomena. A lower number of target phenomena may decrease the risk of overwhelming the individual and/or the risk of introducing a burden that may exceed the capacity of tracking in daily life that potentially could lead to a short duration of the tracking. Although non-obvious, decreasing the number of target phenomena may lead to a more reliable assessment, although less information may be gathered, as there may be a risk that a large number of target phenomena may lead to a shorter duration of tracking of the target phenomena, e.g. a shorter observation period, and/or a lower quality/reliability in the registered observations of occurrences of a target phenomenon.

### Initiation Session

In an embodiment of the present disclosure, the initiation session comprises or consists of conversational therapy, for example therapy according to, at least a part of, the conversational model.

In an embodiment of the present disclosure, the preparatory session comprises or consists of conversations between the individual and the examiner. In a further embodiment of the present disclosure the examiner is a health professional, preferably a mental health professional, such as a therapist, a counselor, a psychologist, a psychotherapist, a psychiatrist, a psychoanalyst, a psychopharmacologist, or a social worker.

In a further embodiment of the present disclosure, the examiner is, at least for parts of the assessment, a conversational software agent. In a specific embodiment of the present disclosure, the examiner is a conversational software agent during the initiation session. The conversational software agent may be based on artificial intelligence, and may further work in combination with a health-professional during the assessment. The conversational software agent may for example ask questions to the individual and use the answers to compile, based on an algorithm, an initial assessment of the state of the individual and/or a suggested plan for the assessment of the suspected condition, which the health professional thereafter may modify, reject or approve.

### Quantitative Hypotheses

In an embodiment of the present disclosure, the set of quantitative hypotheses comprises or consists of one or more expectations, and/or hypotheses, about the occurrences of the target phenomena, such as the dynamics of the suspected condition. It is a preference that at least a part of the quantitative hypotheses are based on the suspected condition of the individual. Typically quantitative hypotheses that are based on the suspected condition of the individual are provided by the examiner, as this typically requires knowledge about the dynamics of the suspected condition.

It may be a preference that the target phenomena is each given any of: a specific value, an approximate value, an upper value, a lower value, a range of values, a null value (absence of occurrence), a non-zero value (occurrence).

The expectation of the occurrences of the target phenomena may alternatively or additionally be described in terms of time, for example at specific time points or time intervals during the day, day of the week, month and/or year, or a combination thereof, and wherein each target phenomena may be given a unique expected values.

The expectation of the occurrences of the target phenomena may alternatively or additionally be described in terms of situations, for example when the individual is performing certain actions, and wherein each target phenomena may be given a unique expected value.

The expectation of the occurrences of the target phenomena may alternatively or additionally be described in terms of events, for example when certain events occur to the individual, and wherein each target phenomena may be given a unique expected value.

The expectation of the occurrences of the target phenomena may alternatively or additionally be described in terms of physical locations, for example when the individual is located at specific physical locations, such as at work or in a car, and wherein each target phenomena may be given a unique expected value.

The expectation of the occurrences of the target phenomena may alternatively or additionally be described in terms of the presence of specific living beings, for example when certain people and/or animals are near and/or interacting with the individual, and wherein each target phenomena may be given a unique expected value. For example some individuals may find relief from interacting with animals, for example dogs or by doing horseback riding.

In an embodiment of the present disclosure, the quantitative hypotheses comprise hypotheses of: the quantitative relationship between a target phenomenon and a source phenomenon (e.g., a relationship between phenomena); the quantitative distribution of the target phenomenon, such as the frequency of the target phenomenon at specific time points of the day and/or at specific locations, and/or the dynamics of these over time.

In an embodiment of the present disclosure, the assessment of the individual comprises exposure therapy, and/or the quantitative hypotheses comprise expected occurrences of the target phenomena in relation to the exposure therapy, such as while being exposed to a challenging situation, such as when the individual takes the bus or goes shopping.

In yet a further embodiment of the present disclosure, the individual registers the quantitative hypotheses by interaction with an interactive system, such as a computational device and/or a conversational software agent. The interactive system may prompt the individual for specific inputs such that the interactive system can formulate the quantitative hypotheses. The interactive system may prompt for input related to the expectations about the occurrences of the target phenomena, and may be based on time, situations, events, physical locations and presence of specific people as discussed elsewhere herein. Furthermore, it is a preference that each target phenomenon is given any of: a specific value, an approximate value, an upper value, a lower value, a range of values, a null value (absence of occurrence), a non-zero value (occurrence). It may further be a preference that the interactive system prompts or allows the individual to input a number of observation distribution and dynamics curves, such as curves showing the frequency or magnitude of a target phenomenon over time. The individual may further be prompted or asked, by the interactive system to point, e.g. by a touch screen, at values related to the frequency of total number of occurrences of each target phenomena, such as high, low or absent, either over the total time period, or for specific intervals.

### Observation Period

During the observation period, the individual is typically expected to register observations, i.e. observations of occurrences of the one or more target phenomena. The observation period runs for a time period which may have a predetermined length or wherein the length is determined by specific criteria, such as until a number of observations have been made, or until the individual or the examiner wants to discontinue the observation period for any reason. The observation period may have any suitable length, depending on for example the suspected condition, and properties of the individual, such as the ability to endure a longer observation period. It may be a preference that the observation period is at least sufficiently long such that enough observations can be registered, or is expected to be registered, such that an updated understanding of the suspected condition of the individual can be derived. The observation period may for example be at least 3 days, or at least 10 day or even at least 60 days. Several properties of the presently disclosed method act to make the individual feel as being in control of the study, for example due to the self-monitoring and the self-registering. Furthermore, the individual may be in control of the stored data, as access to said data may be protected, such as by an access code set by the individual. People other than the individual, including the examiner, may thereby not have access to the data, unless the individual provides access. This further allows the individual to possess a feeling as being in control of the assessment and its progress, which may result in the individual being more prone to complying with the structure, plan and/or definitions of the observation period, such as the observation protocol and/or the intervention protocol, and as set a priori for example in the initiation session.

In an embodiment of the present disclosure, the individual, during the observation period, carries out normal daily tasks, such that the observations at least in part are made in everyday life situations and/or everyday life situations of the individual. The normal daily tasks may thereby be tasks from typical equivalent days of the individual's life, such as a typical workday or a typical non-working day. Alternatively, or additionally, the individual may be exposed to other types of situations (e.g. non-everyday life situations), such as wherein the individual in other ways experience altered behavioral, mental or somatic states or symptoms. For example challenging situations which may not be part of a typical day of the individual. Therefore, in an embodiment of the present disclosure, the individual is exposed to life situations which the individual is expected to experience as challenging. The situations may, in general, not be considered as challenging for an average person. However, the individual may, due to the suspected condition, experience a discomfort when subjected to specific situations.

In a further embodiment of the present disclosure, the observation period comprises exposure therapy. Exposure therapy is a technique in behavior therapy to treat anxiety disorders. Exposure therapy typically involves exposing the individual to triggers of the anxiety source or its context without the intention to cause any danger. Doing so is thought to help them overcome their anxiety or distress.

A specific embodiment comprises at least one of three types of exposure procedures. The first is in vivo or “real life.” This type exposes the individual to actual fear-inducing situations. For example, if someone fears public speaking, the person may be asked to give a speech to a small group of people. The second type of exposure is imaginal, where the individual is asked to imagine a situation that they are afraid of This procedure is helpful for people who need to confront feared thoughts and memories. The third type of exposure is interoceptive, which may be used for more specific disorders such as panic or post-traumatic stress disorder. The individual may confront feared bodily symptoms such as increased heart rate and shortness of breath. Different types of exposure may be used in combination or separately.

In a specific embodiment of the present disclosure, the stored data, such as the stored data of each registered and/or stored observation, comprises or consists of any of force data, location data, orientation data, audio data, present device data, such as present radio-frequency communication device data, light data, heart data, such as heart rate data, galvanic skin response data, temperature data, and/or time data. Force data may include the intensity of the interaction, such as the force by which a button is pressed, the force by which the bodily worn device of the portable data acquisition system is squeezed, and/or the number of times an interaction was repeated consecutively, such as pressing multiple, consecutive times, on a button. Location data may for example comprise GPS coordinates and may further be mapped to physical locations as discussed elsewhere herein. Orientation data may comprise data of the orientation of the device, such as in 3D space, and may be obtained by 6-9 degrees of freedom sensing, the orientation data may for example comprise or consist of angular velocity vector, acceleration vector, magnetic field strength vector, and/or data obtained from an accelerometer, a magnetometer and/or a gyroscope. Audio data may comprise audio recordings and/or audio features that may have been recorded in connection with storing of the observation. The audio recordings may comprise specific information provided by the individual in connection with registering of an occurrence of an observation, but may further comprise audio recording of the surroundings during registering of an occurrence of an observation. The present device data may comprise the present Bluetooth devices, WiFi devices, WiFi hotspots, devices using wireless communication protocols (e.g. WiFi or Bluetooth) present in the vicinity of the portable data acquisition system. Light data may comprise information related to the light at registering of an occurrence of an observation, such as the intensity and wavelength information of the light, e.g. ambient light. Heart data may comprise heart rate data. Galvanic skin response data may comprise data obtained from the individual during registering of an occurrence of an observation. Temperature data may comprise data of temperature measurements during registering of an occurrence of an observation. Any of the stored data may be obtained during registering of an observation of the target phenomenon, or in relation to observation of the target phenomenon, such as during an interval with a predefined time before and/or after said registering of said observation.

### Observing Target Phenomenon

Upon observing, by the individual, an occurrence of one or more target phenomena, the individual is instructed to register an observation. Typically, the individual is to register the observation representing the noticed occurrence of the one or more target phenomena by interaction with a portable data acquisition system. The registration is preferably not a cumbersome nor time-consuming act, instead it may be a preference that the registration of said observation, representing the noticed occurrence of one or more target phenomena can be carried out swiftly, and preferably in a concealed manner. Concealment can for example be provided by wearing the portable data acquisition system under garment, or by having the portable data acquisition system be designed to mimic a commonly worn accessory, such as a watch.

In an embodiment of the present disclosure the information provided to the portable data acquisition system, by the interaction, comprises the observed duration of an occurrence of a target phenomenon. The interaction may be between the individual and a single, or multiple, input components. For the case of a single user input component, the same input component is required to relay multiple types of information, such as information related both to the observation itself of the occurrence of the target phenomenon, and information related to the duration of the occurrence, in addition to any other information provided to the portable data acquisition system. This may be enabled by taking into account other parameters of the interaction, such as the duration of the interaction, the force of the interaction (e.g. force applied to a button or the force by which a part of the portable data acquisition system is squeezed), repetitive interactions (e.g. repetitively pressing a button or repetitively squeezing the instrument for the duration of the interaction, or a number of repetitive interactions that indicate the duration of the observation of the occurrence of a target phenomenon. For example a single interaction, such as a single squeeze of the portable data acquisition system, may indicate a short duration, such as between 0-10 seconds, or between 50-500 milliseconds, or any other predefined time interval, while a repetitive interaction, such as five squeezes of the portable data acquisition system, may indicate a longer duration, such as between 1-2 minutes. Similarly, any number of repetitive interactions may be used to indicate any duration of the observation of an occurrence of the target phenomenon, preferably according to a predefined scheme or pattern.

In an embodiment of the present disclosure the information provided to the portable data acquisition system, by the interaction, comprises the observed magnitude of an occurrence of the target phenomenon. The interaction may be between the individual and a single, or multiple, input component. For the case of a single user input component, the same input component is required to relay multiple types of information, such as information related both to the observation itself of the occurrence of the target phenomenon, and information related to the observed magnitude of the occurrence, in addition to any other information provided to the portable data acquisition system. This may be enabled by taking into account other parameters of the interaction, such as the duration of the interaction, the force of the interaction (e.g. force applied to a button or the force by which a part of the portable data acquisition system is squeezed), repetitive interactions (e.g. repetitively pressing a button or repetitively squeezing the instrument for the duration of the interaction, or a number of times that indicates the observed magnitude of the observation of the target phenomenon. For example a single interaction, such as a single squeeze of the portable data acquisition system, may indicate a low magnitude, such as noticeable but not a significant discomfort, while a repetitive interaction, such as five squeezes of the portable data acquisition system, may indicate a significant discomfort. In this manner, a specific number of repetitive interactions may be used to indicate any magnitude of the observation of the target phenomenon, preferably according to a predefined scheme or pattern.

It should be appreciated that the interaction with the portable data acquisition system may be of any type wherein one or multiple sensors, or other input component, of the portable data acquisition system is able to recognize the interaction as a registration of an observation. Thereby the interaction may be based on vision, audio, or direct physical interaction with the portable data acquisition system. For example the interaction may be based on pressing one or multiple buttons, squeezing a part of the portable data acquisition system, or movement of the portable data acquisition system, such as rotating.

It should further be appreciated, that although the examples provided above relate to the magnitude and the duration of the observation of occurrence of the target phenomenon, the same principle may be applied to any property of the observation of occurrence of the target phenomenon. Preferably, the information provided to the portable data acquisition system, and that is subsequently stored on the device, comprises any information that is relevant for inferring an understanding, such as an updated understanding, of the suspected condition of the individual. The information is preferably provided to the portable data acquisition system by a small number of input components, such as a single button. A small number of input components, i.e. a small number of input modalities, may make the registration of observation of occurrences of the target phenomenon simpler for the individual. Therefore, in a preferred embodiment of the present disclosure, the portable data acquisition system comprises a single user input component, such as a single button or that the portable data acquisition system has a squeezable part, which is able to register several types of information related to the observation of occurrence of the target phenomenon, such as the type of target phenomenon, the duration of the observation, the magnitude of the observation, and/or any state of the individual, such as the mental state, or a combination thereof.

In yet a further embodiment of the present disclosure, the observation protocol comprises instructions for self-monitoring and registering of observations by interaction with the portable data acquisition system in a way that is unique for each target phenomenon, such that each target phenomenon is associated with a unique type of interaction. As mentioned elsewhere herein the interactions may for example differ in terms of the input component, or the specific type of interaction with a specific input component. Thereby, as also mentioned elsewhere herein, a single user input component may allow for multiple types of interactions, and consequently a single user input component may provide different types of information related to the observation of the occurrence of the target phenomenon, to the portable data acquisition system, which thereafter preferably stores said information. The different types of information may for example comprise the type of target phenomenon, the duration of the occurrence, the magnitude of the occurrence, and/or any state of the individual, such as the mental state, or a combination thereof.

It is a preference that information, such as all information, that is provided to the portable data acquisition system is stored, either locally on said portable data acquisition system, or at a remote location, such as on a networked server. In a preferred embodiment of the present disclosure, the portable data acquisition system stores all data that may be useful for inferring an understanding, such as an updated understanding, of the suspected condition of the individual. Preferably this information at least comprises information related to the observation of occurrence of the target phenomenon, such as the time, location, magnitude, duration, and any other values that may be derived from sensors of the portable data acquisition system, either values registered at the time of interaction with the portable data acquisition system (i.e. upon the individual observing the occurrence of the target phenomenon) or at a predefined time, or interval, before or after said registration. Thereby, information related to the environment and/or the individual up until the point of the individual noticing the occurrence of the target phenomenon may be stored on said instrument, and may prove valuable for inferring an understanding of the suspected condition of the individual.

In a specific embodiment of the present disclosure, the time period of the observation period is at least 3 days, such as between 3 days and 90 days. However in alternative embodiments the time period may be shorter or longer without departing from the scope of the present disclosure. It may for example be an advantage to initially have a shorter time period to ensure that for example a vulnerable individual commits to actually trust the assessment method. Thereby in a specific embodiment of the present disclosure, the time period may be on the order of hours.

### Evaluation Session

Following the observation period, the assessment of the suspected condition of the individual typically enters the evaluation session. Typically, the individual has registered a number of observations of occurrences of the target phenomenon, in the form of stored data, either on the portable data acquisition system, or at a remote location, such as a networked server, or both, for syncing of said data.

In an embodiment of the present disclosure, the evaluation session comprises or consists of conversational therapy, for example therapy according to, at least a part of, the conversational model.

In a further embodiment of the present disclosure, the examiner is, at least for parts of the assessment, a conversational software agent. In a specific embodiment of the present disclosure, the examiner is a conversational software agent during the evaluation session. The conversational software agent may be based on artificial intelligence techniques, and may further work in combination with a health-professional during the assessment. The conversational software agent may for example pose questions to the individual and use the answers to compile, based on an algorithm, an initial assessment of the state of the individual and/or a suggested plan for the assessment of the suspected condition, which the health professional thereafter may modify, reject or approve.

In an embodiment of the present disclosure, the recollection of the observation period is a subjective recollection of the observed occurrences of the target phenomenon, by the individual. The recollection may be subjected to a measurement bias, such as recall bias and/or reporting bias.

Recall bias as used herein refers to a systematic error caused by differences in the accuracy or completeness of the recollections retrieved (“recalled”) by human subjects regarding events or experiences from the past.

Reporting bias as used herein refers to selective revealing or suppression of information by human subjects (i.e. individuals).

In a further embodiment of the present disclosure, the recollection comprises or consists of any information related to the observed occurrences of the target phenomenon. For example the evaluation session may comprise a recollection, by the individual, comprising (as a combination of all target phenomena, or for each target phenomenon) the frequencies, the total number of observed occurrences, the durations of the observed occurrences, and/or the intensities of the observed occurrences, as a sum over the entire period, or as a distribution over time.

In a further embodiment of the present disclosure, the recollection further comprises a mapping of circumstances, such as situations, locations, events, and other people present, that may have led to the triggering of the occurrences of the target phenomenon. It may be beneficial to not just consider the circumstances during the observation of occurrence of the target phenomenon, but additionally or alternatively, consider what may have caused the target phenomenon. Therefore, the recollection may comprise information related to the situations, the physical locations of the individual, events, specific people near the individual, and/or specific people interacting with the individual, that may have occurred before the observation of the occurrence of the target phenomenon, such as a predetermined time period before said observation.

In an embodiment of the present disclosure, prior to acquisition of the stored data, consent to acquire the stored data is obtained from the individual, such as by the examiner. People other than the individual, including the examiner, may thereby not have access to the data, unless the individual provides access. This may enable the individual to attain a sense of being in control of the assessment and its progress, which may result in the individual being more prone to complying with the structure, plan and/or definitions of the observation period, such as the observation protocol and/or the intervention protocol, and as set a priori for example in the initiation session.

In a specific embodiment of the present disclosure, the acquisition of the stored data is access-controlled. The access-control may require a type of authentication, such as an access key, for giving permission to acquire the stored data. The authentication may be a one-time use alphanumeric key, provided by the individual. The individual may have a number of access keys, or may continuously be able to generate new access keys. It is a preference that the access keys are one-time use, and thereby, that following a first acquisition of the data a new access key may be needed for a second acquisition of the data, such as new data that have been stored between the first and second acquisition period. The authentication may comprise any type of identification for controlling the acquisition of the stored data, and may include single factor authentication, two factor authentication (2FA), multi-factor authentication, authentication method protocols, and/or API authentication, such as HTTP basic auth, API keys, and/or OAuth.

### Quantitative Comparison and Visual Overview

In a preferred embodiment of the present disclosure, the assessment comprises the generation of a visual overview of any of: the stored data, the recollection, and/or the quantitative hypotheses. The visual overview may be provided in the form of a timeline and/or a heat map, such that patterns in the data can be identified. It is a preference that the visual overview is presented to the examiner and/or the individual during the evaluation session. The visual overview may be used during the evaluations session, such as in a dialogue about the observed occurrences of the target phenomenon during the observation period, for identification of discrepancies between the stored data, the recollection, and/or the quantitative hypotheses, or for making quantitative inferences. Discrepancies may for example comprise the number of observations (part of the stored data) in relation to the expected number of observations (part of the quantitative hypotheses) and/or in relation to the recollection.

In a further embodiment of the present disclosure, the quantitative comparisons comprise a visual overview of any of the stored data, the recollection, and/or the quantitative hypotheses.

The visual overview may be provided in the form of one or multiple histograms, one or multiple calendar views, and may further comprise summary statistics on any, or multiple, of the stored data, the recollection, and/or the quantitative hypotheses. The visual overview may for example comprise the number of observations, the duration of the observations, the average number of observations per day, the average observations per week, the average number of observations per weekday, the average number of observations per day each week, the average number of observations per day each week, the distribution of observations on different hours of the day (e.g. night, morning, noon, afternoon, late afternoon, evening), or similar values for the recollection, the quantitative hypotheses, or other parts of the stored data. The visual overview may be provided as combined values and/or statistics for all target phenomena, or may be additionally or alternatively, be provided for each target phenomenon separately.

In an embodiment of the present disclosure, the location data and/or the time-stamp are used to bin the observations to a number of places. The location data may for example be provided in the form of GPS coordinates, and may either upon registration of the observation, or upon acquisition of the information, be transformed into the places. Places may be physical locations, such as the home of the individual, or the workplace of the individual, and may be used as part of the visual overview. For example the frequency, duration, or magnitude may be mapped to the different places. Preferably, the analysis of the stored data is carried out such that irregularities, such as abnormal values of occurrences of one or more places can be identified. In this sense, an observation may be normalized, to the time spent at a specific location, such that the frequencies can be derived.

In a further embodiment of the present disclosure, the places have pseudonymized or randomized labels, such as A, B and C. By masking the true locations and/or the places, and identifying places using pseudonymized or randomized labels, the individual may feel more comfortable with registering observations, as the exact place will not be revealed, such as to the examiner. Instead, the visual overview, or any other type of data analysis, may be based on synthetic labels of the places. In an alternative embodiment of the present disclosure, the places are labeled corresponding to the type of physical locations, such as workplace, home and commute.

### Continued Progress

In an embodiment of the present disclosure, the continued progress of the assessment of the suspected condition, following the evaluation session, comprises any, or a combination, of: completion of the assessment; continuing the assessment with interventions; and updating any of i. the set of quantitative hypotheses, ii. the observation protocol and/or iii. the intervention protocol, and repeating any of steps i-iii, at least partly.

Preferably, the continued progress of the assessment of the suspected condition is based on the results of the quantitative comparison. The outcome of the quantitative comparison may lead to a sufficiently good understanding of the condition of the individual, such that it is suitable to complete the assessment, the individual may thereby be considered to have been treated, or following the assessment the individual may be equipped with the necessary means for dealing with the condition or for being treated. For example, the individual may be referred to another health and/or medical professional, or the individual may be provided with tools and/or medication in order to decrease the discomfort experienced by the individual, or the individual may be instructed to avoid specific circumstances that are associated with a high risk of occurrences of prominent and/or detrimental phenomena. Alternatively, or additionally, the individual may be instructed to carry out interventions upon observation of a target phenomenon. These may be carried out after completion of the assessment or carried out as part of a continued assessment. Furthermore, the assessment may be repeated, at least in part. Repeating, at least in part, the assessment may be done while updating any of i. the set of hypotheses, ii. the observation protocol and/or iii. the intervention protocol. Alternatively, parts of the assessment may be repeated for acquisition of additional data, in such cases it may not be required to update any of i. the set of hypotheses, ii. the observation protocol and/or iii. the intervention protocol, although it may be preferred. In a specific embodiment of the present disclosure, any part of the assessment may be repeated at any time. For example, the initiation session may be repeated a number of times before progressing to the observation period and/or the evaluation session. Alternatively or additionally, the observation period may be repeated a number a number of times before the assessment progresses to the evaluation session. In a specific embodiment, any of the evaluation session and the observation period may be repeated following completion of the initiation session and/or the observation period. Alternatively or additionally, the evaluation session may be repeated following completion of the evaluation session. When repeating any part of the method parameters may be updated in order to improve the assessment.

### Intervention Protocol

In one embodiment of the present disclosure, the individual is instructed to carry out one or more types of interventions, upon observation of an occurrence of a target phenomenon. Each type of intervention may be associated with a specific target phenomenon. Intervention may be, but not limited to, breathing exercises, relaxation exercises, mindfulness exercises, and/or exercises for changing thought patterns, such as behavioral exercises.

In an embodiment of the present disclosure, the initiation session further comprises definition of an intervention protocol, comprising or consisting of instructions for how the individual is to perform a self-administered intervention. The intervention protocol may be, at least in part, based on the suspected condition of the individual and/or the quantitative hypotheses.

In a further embodiment of the present disclosure, the intervention protocol comprises, or consists of definitions and/or instructions for when the individual is to perform the self-administered intervention, such as upon when the individual observes an occurrence of the target phenomenon and/or upon obtaining an intervention prompt, such as from the portable data acquisition system.

In yet a further embodiment of the present disclosure, the portable data acquisition system is configured to provide the intervention prompt at any of: after a predetermined number of registered observations, at regular time intervals, at random time intervals, at specific time points during the day, below, at or above a specific frequency of observations, at certain locations and/or after observations with an magnitude below, at or above a predetermined threshold.

In yet a further embodiment of the present disclosure, the intervention protocol comprises instructions of how the individual is to register completion of an intervention, upon each completion of an intervention. The registration may be performed by an input component that is separate from the input component for registering observations, such as a separate button, however it may be a preference that the input component is the same for both registering the completion of an intervention and registering observations. As discussed, elsewhere herein, there are several strategies for using a single user input component while simultaneously being able to provide the portable data acquisition system with different types of signals. For a single button as the input component (user input component), the duration, the force, the number of consecutive presses, or the specific sequence of presses (e.g. consecutive longer, shorter, harder, and/or softer presses), may for example be used to input specific codes with a predetermined meaning. Instead of a button, interaction with the portable data acquisition system may rely on squeezing said instrument. Squeezing may be carried out in the same way for providing different types of signals with different meanings, i.e. any of the duration, the force, the number of consecutive presses, or the specific sequence of presses (e.g. consecutive longer, shorter, harder, and/or softer presses), may be altered. The portable data acquisition system may further rely on gesture-based input, for example by the use of accelerometers, and thereby various gestures can be used in order to provide said instrument with different types of signals.

Portable Data Acquisition System

In an embodiment of the present disclosure, the portable data acquisition system comprises one or more input devices on the bodily worn component and/or the processing and display component. The input device is preferably designed such that an individual can interact with the portable data acquisition system/input device. For example, the input device may be provided in the form of one or more buttons, one or more pressure sensors, one or more touch screens, one or more fingerprint sensors, and/or one or more microphones. Further details regarding the input device and how it is used to register data will be described in greater detail hereinbelow.

The individual may interact with the bodily worn component and/or the processing and display component in such a way that the portable data acquisition system is provided with a signal. The interaction may depend on what type of input device the portable data acquisition system is equipped with, and the programming of the portable data acquisition system, i.e. instructions stored in a random-access and/or a persistent memory on the portable data acquisition system. The portable data acquisition system may be programmed to be able to retrieve multiple types of signals. These signals may be provided through multiple input components, or alternatively a single user input component. For a single button as the input device (user input device), the duration, the force, the number of consecutive presses, or the specific sequence of presses (e.g. consecutive longer, shorter, harder, and/or softer presses), may for example be used to input specific codes with a predetermined meaning. Instead of a button, interaction with the portable data acquisition system may rely on squeezing said instrument. Squeezing may be carried out in the same way for providing different types of signals with different meanings, i.e. any of the duration, the force, the number of consecutive presses, or the specific sequence of presses (e.g. consecutive longer, shorter, harder, and/or softer presses), may be altered. The portable data acquisition system may further rely on gesture-based input, for example by the use of accelerometers, and thereby various gestures can be used in order to provide said instrument with different types of signals.

In a further embodiment of the present disclosure, the portable data acquisition system is configured for multiple different types of interactions, by a single or by multiple input devices. Preferably, the different types of interactions results in different signals being provided to the portable data acquisition system. It is a further preference that different types of signals are used in order to store different types of information by the portable data acquisition system, such as in a memory on the portable data acquisition system or on a remote server, such as a networked server, or both for syncing of said data. The portable data acquisition system may thereby further comprise a component for communication with a remote server, such as wireless radios and/or antennas for wireless communication. Different types of interactions may thereby be used when the individual wants to register different types of events, for example upon observation of occurrence of a specific target phenomenon, or upon completion of an intervention according to the intervention protocol. They have preferably been instructed to interact with the portable data acquisition system such that each specific event that needs to be registered, is registered by its dedicated type of interaction with the portable data acquisition system. Furthermore, as each specific type of interaction preferably renders a specific type of signal, the portable data acquisition system, such as the processing unit of the portable data acquisition system, is able to store data comprising information related to the type of event that has occurred. The stored data may comprise information that is based on, or consists of, additional input received from sensors embedded in the portable data acquisition system. Furthermore, the stored data may comprise information about the time when said data was stored or when the event occurred and/or observation was registered.

In an embodiment of the present disclosure, the portable data acquisition system is an instrument dedicated to the registration of observations and/or for transferring data collected during registration of observations, such as during the observation period. Thereby, said portable data acquisition system may comprise or consist of components for registering observations and components for communication, such as wireless communication and/or wired communication. Said components for registering observations may comprise a memory, such as a local memory for storing data related to registrations of said observations.

It should be noted that data collection in the past decade increasingly has come to rely on smartphones and smartwatches. However, these types of devices are not always suitable instrumentations for high temporal resolution, in-the-moment self-tracking of subjective experience. Specifically, the data collection may be hampered by interaction modalities that are too time-consuming and cumbersome in everyday life situations. Said devices are multi-purpose and may distract individuals with other information during the course of making an observation of an occurrence of a target phenomenon. A visual user interface discloses, to others, that an individual is doing data collection which might be disruptive in social situations and potentially stigmatizing. A visual user interface also requires additional attention (in particular visual attention) by the individual, which makes it more cumbersome and time consuming to register observations. Also, with smartphones and smartwatches there is an associated daily burden of keeping them charged and operational. Further, the monetary value of these devices exposes socially vulnerable individuals to a risk of e.g. assault.

In an embodiment of the present disclosure the stored data, e.g. the interaction data, may be based on the type of interaction. The interaction may be between the individual and a single, or multiple, input components. For the case of a single user input component, the same input component may be required to relay multiple types of information, for example information related both to the observation of the occurrence itself of the target phenomenon, and information related to the magnitude of the occurrence. This may be enabled by taking into account other parameters of the interaction, such as the duration of the interaction, the force of the interaction (e.g. force applied to a button or the force by which a part of the portable data acquisition system is squeezed), repetitive interactions (e.g. repetitively pressing a button or repetitively squeezing the instrument for the duration of the interaction, or a number of times that indicates the magnitude of the observation of the target phenomenon. For example a single interaction, such as a single squeeze of the portable data acquisition system, may indicate a low magnitude, such as noticeable but not a significant discomfort, while a repetitive interaction, such as five squeezes of the portable data acquisition system, may indicate a significant discomfort. In this manner, a specific number of repetitive interactions may be used to indicate any magnitude of the observation of the target phenomenon, preferably according to a predefined scheme or pattern.

In a preferred embodiment of the present disclosure, the portable data acquisition system comprises a single user input component. A single user input component, may make the procedure of registering observations of occurrences of target phenomena and other types of events simpler for the individual, and may further make it possible to design the portable data acquisition system sufficiently small such that it can easily be concealed, for example as being worn as part of an attire, around the wrist, similar to a watch, or around the neck similar to an pendant.

In a further embodiment of the present disclosure, the portable data acquisition system is configured for multiple different types of interactions, and wherein each target phenomenon and/or, the completion of an intervention, is associated with a unique type of interaction. In yet a further embodiment of the present disclosure, the portable data acquisition system, upon interaction, automatically stores data, such that each interaction is associated with unique interaction data.

In a further embodiment of the present disclosure, the interaction data and/or the stored data comprises contextual data, such as selected from the list including: force by which the button is pressed, time-stamp, location data, temperature data, orientation of the device, audio recording, such as audio features, present Bluetooth devices, present WiFi devices/hotspots, devices using wireless communication protocols (e.g. Bluetooth or WiFi) present in the vicinity of the portable data acquisition system, intensity and other features of the ambient light.

In a further embodiment of the present disclosure, the interaction data and/or the stored data comprises information related to physiological measurements of the individual, such as heart rate and heart rate variability (HRV) measurements, galvanic skin response (GSR) measurements, motion sensors, and/or temperature measurements.

In a further embodiment of the present disclosure, the observation information is stored locally on the portable data acquisition system, or on a networked server, or a combination thereof.

In a further embodiment of the present disclosure, the portable data acquisition system is configured to provide feedback upon interaction, such as haptic feedback, auditory feedback and/or visual feedback. Feedback may for example make the individual aware, that the interaction has been detected by the portable data acquisition system, and that data has been stored in a memory of the portable data acquisition system. Wherein the stored data may for example include interaction data related to the type of interaction, and contextual and/or physiological data, that may have been provided by sensors of the portable data acquisition system.

In an embodiment of the present disclosure, the portable data acquisition system is a computational device, such as a mobile phone, a tablet, or a computer. In a specific embodiment of the present disclosure, the portable data acquisition system is wearable, such as being suitable for being worn on the wrist, around the neck, and/or attached to the belt. Consequently, the portable data acquisition system may comprise a wristband, a lanyard, and/or a belt mount, or any type of attachment to the body, attires, or devices worn by the individual. It may be a preference that the portable data acquisition system is positioned at, or near, the location of the body where the individual is expected to notice occurrences of the target phenomenon. For example, if the individual is to register chest pains, the portable data acquisition may be advantageously positioned near the chest, consequently, said instrument may be provided as a pendant.

In a further embodiment of the present disclosure, the portable data acquisition system comprises a power source, a memory, such as a persistent or a random-access memory, and a processing unit.

The portable data acquisition system may further comprise a communication component, such as wireless communication radios and/or antennas, for transferring of data, such as the stored data. Thereby, the communication component may be used for acquisition of the stored data, for example during the evaluation session. Furthermore, the portable acquisition instrument may comprise a display, such as a monitor. The display may further act as an input component, in the form of a touchscreen.

In a further embodiment of the present disclosure, the portable data acquisition system is reconfigurable and comprises an input component, for programming and/or reprogramming instructions stored in the memory of the portable data acquisition system.

The present disclosure will in the following be described in greater detail with reference to the accompanying drawings. The drawings are exemplary and are intended to illustrate some of the features of the presently disclosed method for assessment of one or more suspected conditions of an individual by an examiner, and are not to be construed as limiting to the present disclosure.

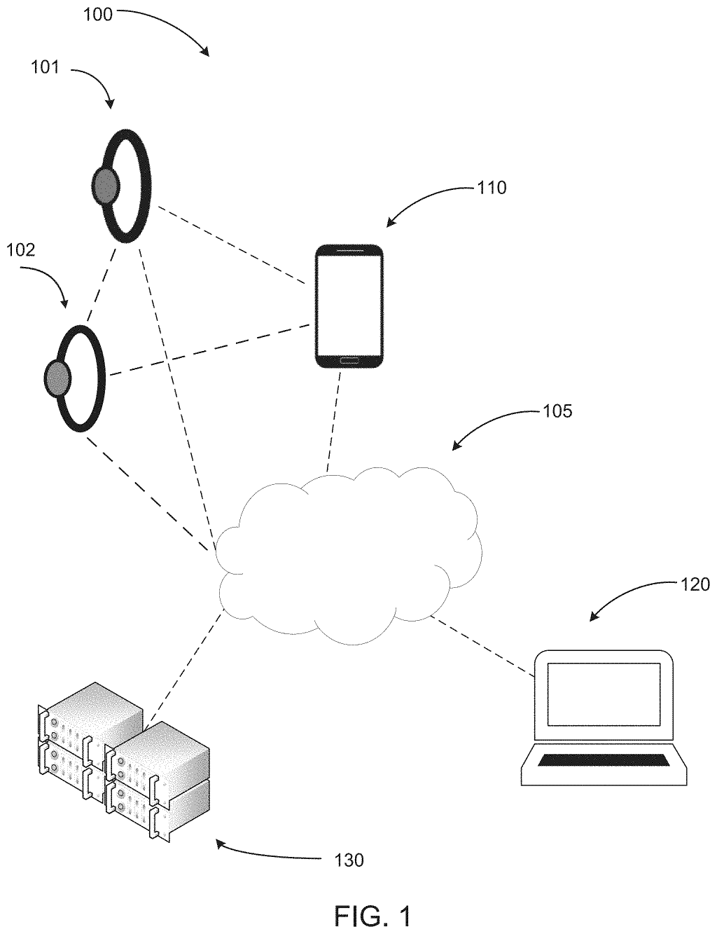

Referring now to the drawings, FIG. 1 depicts a system, such as an illustrative network of components for quantitative assessment of a health condition, according to embodiments shown and described herein. As illustrated in FIG. 1, a network (105) may include a wide area network (WAN), such as the Internet, a local area network (LAN), a mobile communications network, a public service telephone network (PSTN), a personal area network (PAN), a metropolitan area network (MAN), a virtual private network (VPN), and/or another network. The network (105) may generally be configured to electronically connect one or more computing devices and/or components thereof. Illustrative devices and components may include, but are not limited to, a data acquisition system (100) that includes a bodily worn data acquisition device (101), an intervention system (102), and/or a processing and display component (110) (e.g., a smartphone), an assessment device (120), and an external device (130). It should be understood that the processing and display component (110) may be omitted in some embodiments.

The system (100) includes a data acquisition device (101) and an intervention system (102) and may in a preferred embodiment be the same physical system (a bodily worn device with a single, main button and haptic feedback mechanism, such as a wristband). Another example embodiment may involve two separate physical devices, where one is the data acquisition system for obtaining input from the user (a device with one main button) and the other is the intervention system (e.g. a smartwatch that could provide both haptic and audible feedback to the user).

Each of the components of the portable data acquisition system (100) (e.g., the bodily worn data acquisition device (101), the intervention system (102), and/or the processing and display component (110)) may generally be used as an interface between a user (e.g., a subject, a patient, an individual that is being assessed, or the like) and the other components connected (e.g., communicatively coupled) to the network (105). Thus, the portable data acquisition system (100) may be used to perform one or more user-facing functions, such as receiving one or more inputs from a user or providing information to a user, as described herein. The components of the portable data acquisition system may be communicatively coupled to one another and/or may be communicatively coupled to the other components of the network (105), as indicated by the dashed lines in FIG. 1.

The assessment device (120) may be used as an interface between a user and the other components connected to the network (105) and/or as a component that receives information and/or provides instructions. Thus, the assessment device (120) may be used to perform one or more user-facing functions, such as receiving one or more inputs from a user or providing information to the user, as described in greater detail herein. The external device (130) may receive data from one or more sources, store the data, and provide information from certain portions of the data to various components.

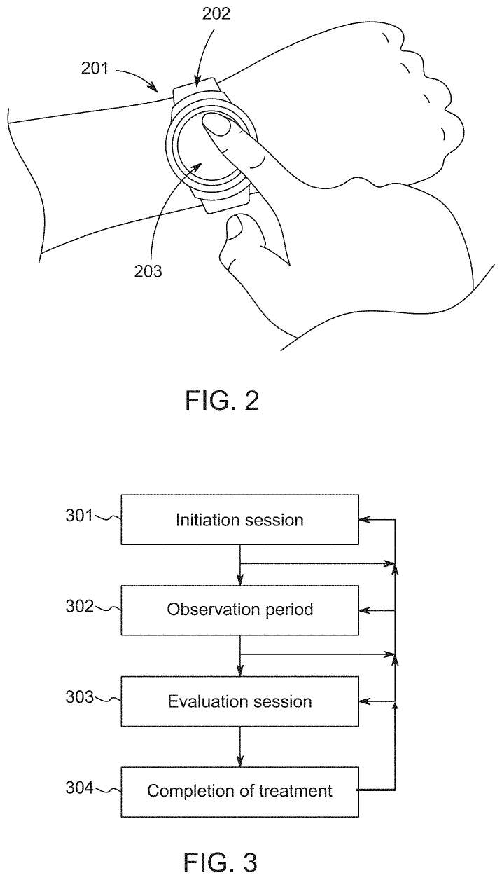

It should be understood that while the assessment device (120) is depicted as a personal computer and the external device (130) is depicted as a server, these are nonlimiting examples. More specifically, in some embodiments, any type of computing device (e.g., mobile computing device, personal computer, server, etc.) may be used for any of these components. Additionally, while each of these computing devices is illustrated in FIG. 1 as a single piece of hardware, this is also merely an example. More specifically, each of devices depicted in FIG. 1 may represent a plurality of computers, servers, databases, components, and/or the like. FIG. 2 shows an exemplary bodily worn data acquisition device (201) of the portable data acquisition system (100), which in this case is provided with a wrist strap (202) such that the individual can wear said bodily worn component around the wrist. The bodily worn component comprises input component (203) for registering observations, according to a specific embodiment of the present disclosure. In this exemplary embodiment, the bodily worn component is provided as a one-button-tracking device, wherein the device comprises a single user input component, in the form of a button, for registering observations. As this exemplary bodily worn component is intended to be worn by the individual at all times, on the wrist, the bodily worn component is always available for the individual. Thereby, the individual can easily register observations by interacting with the bodily worn component, such as by pressing the button. As disclosed elsewhere herein, in addition to registering the observation of occurrence of a target phenomena, the same input component may further be used for registering parameters of the observation, e.g. parameters of the one or more target phenomena, such as the duration of the observation, the magnitude of the observation, and the type of target phenomena. While the bodily worn component may be configured and/or designed such that it can be concealed or have the appearance of a common accessory, such as a watch, the bodily worn component may capture a wealth of information upon registering an observation. It is a preference that the bodily worn component at least registers data, such as interaction data, related to the time point of the individual's interaction with the instrument in connection with an observed occurrence of the target phenomena. The interaction data may for example comprise measurements of aspects of the individual's physiological state, and/or contextual information, such as GPS coordinates, orientation of the instrument, temperature data, audio recordings and present active wireless communication devices. Upon registering an observation, it is a preference that the bodily worn component stores data related to the observation, such as contextual information and/or physiological information of the individual. Preferably said data is stored in the memory of the bodily worn component, and furthermore continuously synced with a remote server. While the depicted bodily worn component is configured to register observations when an individual presses a button, the bodily worn component can be configured in many different ways for this task, as disclosed elsewhere herein. It should also be understood that some of the functionality described herein with respect to the bodily worn component may alternatively or additionally be provided by one or more other components of the portable data acquisition system, such as the processing and display component.

FIG. 3 shows a flow diagram outlining a typical method for assessment of a suspected condition of an individual, according to an embodiment of the present disclosure. The exemplary method comprises an initiation session (block 301), an observation period (block 302), and an evaluation session (block 303). The initiation session typically comprises or consists of conversation between the individual and an examiner. The conversations may for example comprise conversational therapy, such as therapy according to the conversational model. In the initiation session it is a preference that an observation protocol is defined comprising or consisting of instructions for how the individual is to self-monitor and register observations in the form of stored data, by interaction with a portable data acquisition system, upon each occurrence of the target phenomenon is observed by the individual. During the observation period the individual may be instructed to carry out self-monitoring and registering observations of the target phenomena on the portable data acquisition system, such as according to the observation protocol. The evaluation session may also comprise conversation, such as between the individual and the examiner. It is a preference that the stored data, of the portable data acquisition system, is acquired and a recollection of the observation period is made by the individual. Thereafter one or more quantitative comparisons are typically carried out between any of the stored data, the quantitative hypotheses, and/or the recollection. Based on the quantitative comparisons, the examiner may infer based on the quantitative comparison, an understanding of the suspected condition of the individual. Said understanding may be used to conclude a continued progress of the assessment of the individual. The continued progress, following the evaluation session, may either be the completion of the assessment (block 304) or updating any of i. the quantitative hypotheses, ii. the observation protocol and/or iii. the intervention protocol, and repeating the initiation session, the observation period and/or the evaluation session, at least partly.

The observation protocol and intervention protocol needs to be accessible to the data acquisition system and the intervention system in order to adapt the behavior of the systems (in real time) according to the protocols. For example, a user may be asked to perform an intervention when a number of observations has been made within a certain time frame (one hour). Thus the data acquisition system needs to be able to monitor this threshold and signal the intervention system. Or conversely, the intervention system has to know the observation protocol, too, to be able to interpret incoming interaction data packages and act based on the intervention protocol.

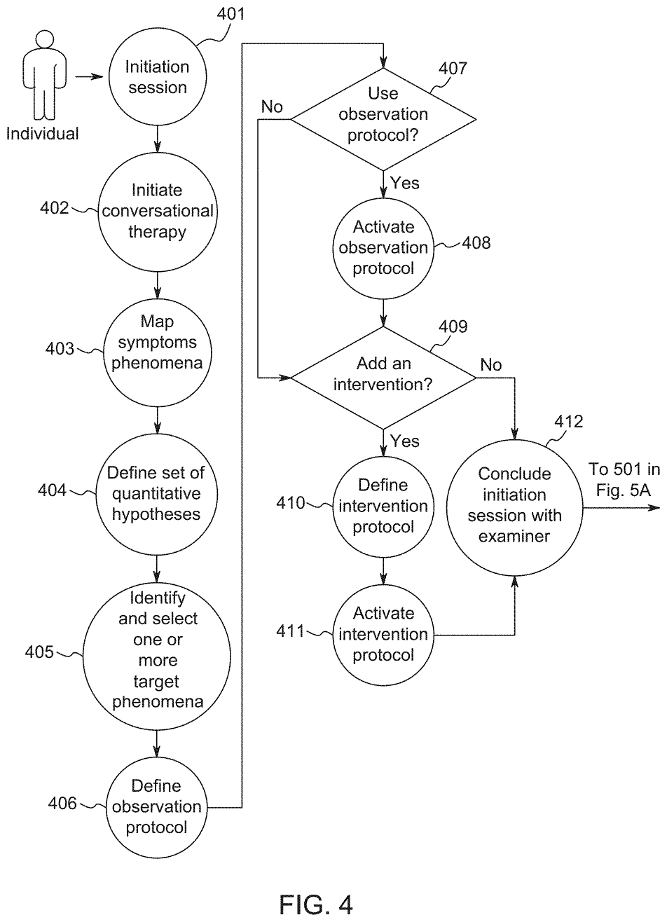

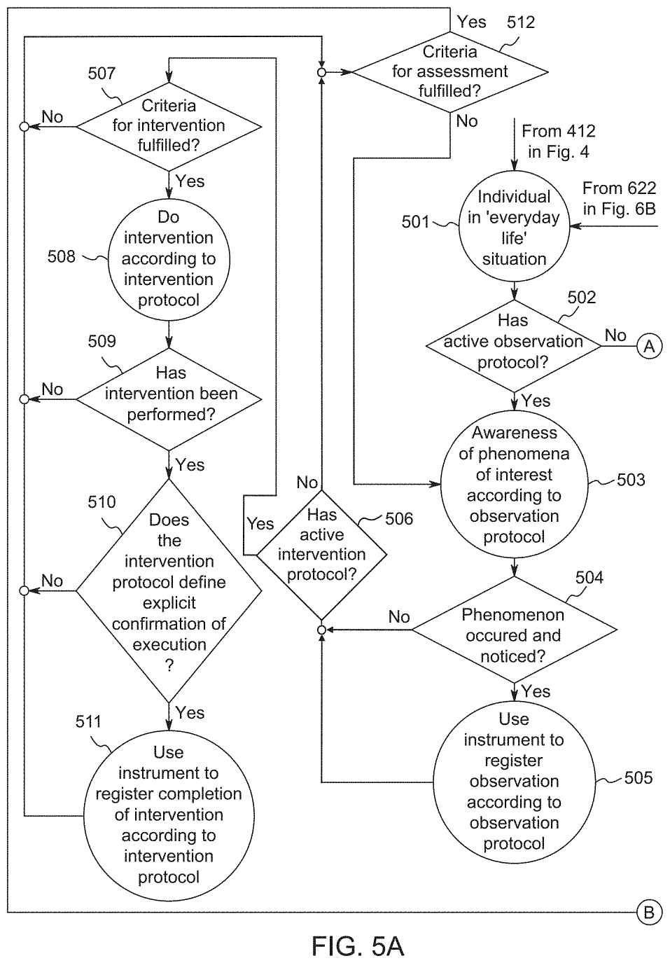

FIG. 4 shows a flow diagram outlining an initiation session, according to a specific embodiment of the present disclosure. The exemplified method for assessment of one or more suspected conditions of an individual by an examiner is typically initiated in an initiation session (block 401), said initiation session typically comprises conversation, such as conversational therapy (block 402), between the individual and the examiner. It is furthermore a preference that the initiation session comprises the identification of one or more features of the suspected condition (block 403). Said identification of the features of the suspected condition may be a collaborative effort, between the examiner and the individual. Furthermore, one or more features of the suspected condition are typically identified, and one or more subject-specific phenomena of presentation. By the subject-specific phenomena of presentation one or more target phenomena may be defined, identified and/or selected (block 405). Said session further comprises the generation of one or more quantitative hypotheses (block 404), typically comprising or consisting of a number of expectations about the occurrences of the target phenomena, and may for example be hypotheses about i. the quantitative relationship between a target phenomenon, and a source phenomenon and/or ii. the quantitative distribution of the target phenomenon, such as the frequency of the target phenomenon at specific time intervals of the day and/or at specific locations, and/or the changes of these over time.

Defining a set of quantitative hypotheses may be completed such that a quantitative hypothesis is formulated as statements about the expectations about the occurrence, non-occurrence, temporal dynamics, and/or pattern of occurrences of a target phenomenon specified in the observation protocol. Given a certain set of conditions of contextual parameters a statement about the expectations of the occurrences of a target phenomenon may be formulated in quantitative terms. Typically, quantitative hypotheses translate from the time domain to the incidence domain (e.g., number of occurrences in a fixed time duration such as a number of observations per day). A quantitative hypothesis is a formalization of possible quantitative relations between target and source phenomena and is used by the assessment system to be able to execute assessments algorithmically. The process includes formulation of one or more questions about a suspected condition posed with respect to an identified target phenomenon, the observations of which are likely, given the subject-specific presentation of the suspected condition, to yield a time series of observations of the target phenomenon to use for the evaluation of the quantitative hypothesis and subsequent obtainment of answers for said one or more questions. Examples of generic quantitative relations include: Incidence of target phenomenon stays constant over time, Incidence of target phenomenon increases or decreases over time, Incidence of target phenomenon is higher or lower compared to baseline at certain time intervals and/or at certain places and/or with or without certain people present and/or with the individual being exposed to certain external stimuli, and/or intake of food or other substances, and/or under certain environmental conditions, incidence of target phenomenon exhibits sudden changes. One specific example may indicate that there are more observations of the target phenomenon in the afternoon. Accordingly, the system in order to evaluate an associated quantitative hypothesis would need to hold definitions of what the target phenomenon is, what “afternoon” is (i.e., a time interval that is recurring on a given temporal scale), and what “more” is (i.e., how to perform a greater than operation on the relevant variable). The procedure for specifying a quantitative hypothesis includes the following steps:
1. Define time period of interest (statically or parametric/rule-based)
2. Select target phenomenon
3. Select source phenomena
4. Specify the criteria for the occurrences and/or incidences and/or derivatives of the incidences of the target phenomenon to include respectively exclude
5. Specify the criteria for the occurrences and/or incidences and/or derivatives of the incidences of the source phenomena to include respectively exclude
6. Specify the quantitative relation between the included occurrences of the target and source phenomena as specified by the criteria in 4) and 5)
7. (Optional) Specify the plausibility of the quantitative hypothesis
8. Persist the quantitative hypothesis

Additional examples of definitions of quantitative hypotheses include: i. If at work and the boss is present then the incidence is higher than the incidence in situations at work where the boss is not present and ii. The incidence of the target phenomenon will trend downwards during the course (time period of interest) of the treatment meaning that the slope of the regression of the incidence for the time period of interest is negative. For example, the incidence of the target phenomenon (symptoms of withdrawal) will decrease sometime in the future (but not immediately and it might initially increase) as the source phenomenon (intake of antidepressant medicine) is decreased (phased out). It could potentially be specified explicitly when the quantitative hypothesis is expected to be confirmed by the observations time series.

The initiation session further comprises the definition of an observation protocol (block 406), comprising or consisting of instructions for how the individual is to self-monitor and register observations in the form of stored data, by interaction with a portable data acquisition system, upon each occurrence of the target phenomenon is observed by the individual. The observation protocol may be defined according to block 406 via a series of inputs received from a user. For example, a user may provide inputs via a user interface to a series of queries that are presented to the user as a means of defining the observation protocol. In some embodiments, the series of queries may be presented to the user via the user interface such that the user is able to select a phenomenon, select an encoding in terms of an interaction pattern for the phenomenon among a list of available encodings, such as, for example, a threshold for the temporal distance between interactions, a threshold for a duration of an interaction, a duration of an interaction, a number of consecutive interactions, an applied force, and/or an orientation and/or displacement of the data acquisition system (or component thereof). In some embodiments, human-readable instructions and/or machine-readable instructions for the selected encoding pattern may be defined. The chosen encoding is then removed from a set of available encodings, the phenomenon definition may be persisted, and while target phenomena are not encoded, the steps may be repeated. There may only be one active observation protocol for an instrument at a given time. Illustrative examples of observation protocol include the following:

### EXAMPLE 1

1. Target phenomenon: Arousal experienced by the individual as tension in the chest muscles
2. Interaction pattern encoding: Press the button once each time the tension is experienced. If the tension remains for a longer duration press the button again. Repeat this until the tension is gone.
3. Human-readable instructions to the individual are given similar to the wording of the previous bullet.

### EXAMPLE 2

Pass 1:
1. Target phenomenon: Flashback of a traumatic experience
2. Interaction pattern encoding: Press the button for less than 500 ms to record a flashback observation.

Pass 2:
1. Phenomenon: Ruminating thoughts
2. Interaction pattern encoding: Press the button for more than 500 ms to record a rumination observation

Human-readable instructions: Provided as stated in the research protocol of the study the individual is participating in.

Typically the examiner decides whether the observation protocol is to be activated (block 407). While activation (block 408) of the observation protocol may be advantageous for specific applications, it may be preferred to not activate the observation protocol until later stages of the assessment, or even not activate the observation protocol at all. It is a further preference that the assessment comprises the consideration of addition of interventions (block 409). The interventions, if they are to be used, are typically defined in an intervention protocol (block 410) and may comprise instructions on how the individual is to carry out one or more types of interventions, upon observation of an occurrence of a target phenomena.

Defining an intervention protocol according to block 410 may specify how the user is to interact with the data acquisition system both in a machine-interpretable way, but also in a human-understandable way. Interventions may be, for example, human-triggered vs. system-triggered, human-performed interventions, and machine-performed interventions. The process may include, for example,

1. Select an intervention

2. Define intervention trigger criteria
(Optional) user criteria
(Optional) system criteria

3. Define intervention execution
(Optional) user performs intervention
   i. (Optional) Define human-readable instructions for the selected intervention
(Optional) system performs intervention
   i. Define the intervention scheme

4. Define intervention completion
(Optional) If an explicit confirmation of execution of an intervention is needed, then this is defined as an encoding of an interaction pattern when defining the observation protocol.
   i. Remove the chosen encoding from the set of available interaction pattern encodings

5. Persist the intervention protocol

6. While there are interventions not yet defined repeat step 1-5.

### Examples

1. User driven intervention:
a. defined trigger that is observable/determinable by the user (e.g. high arousal or a panic attack)
b. a defined intervention to be performed by the user (a mental exercise)
c. an interaction encoding to confirm the execution of the intervention (press button three times)

2. System driven intervention:
a. trigger condition determinable by the system (increased incidence above a set threshold of observations of the target phenomenon during the past hour)
b. a feedback pattern (vibrate in a pulsating pattern of 0.1 Hz for 90 seconds)
c. (Optional) confirmation (using an interaction encoding) that the user noticed the intervention was performed by the system (press button three times)

3. Hybrid: (interplay between user and system while executing an intervention)
a. trigger condition determinable by the system (increased incidence above a set threshold of observations of target phenomenon during the past 4 hours)
b. the user inputs the stress level (press 1-5 times to indicate stress level)
c. based on the user input the system adjusts the intervention scheme parameters,
e.g.
   i. based on the indicated stress level the intervention duration is adjusted
   ii. the intervention commences with haptic feedback (on the data acquisition system) every 3 seconds for 60 seconds while the user performs a mental exercise to calm down from a medium level of arousal
d. confirmation (using a selected interaction encoding) that the user has performed the mental exercise (press button three times)

It should be noted that, similar to the observation protocol, the presently explained method is not limited to the use of an intervention protocol. Instead the intervention protocol may not be used, or the intervention protocol may be activated at a later stage of the assessment. Each type of intervention may be associated with a specific target phenomenon. The intervention may be, but not limited to, breathing exercises, relaxation exercises, mindfulness exercises, exercises for changing thought patterns. Following definition of the intervention protocol it may be activated (block 411). Thereafter, the initiation session may be concluded (block 412), and the assessment may continue with the observation period. However, although not shown, the assessment may comprise a repetition of the initiation session, wherein said repetition may involve a modification of any of the steps of the initiation session.

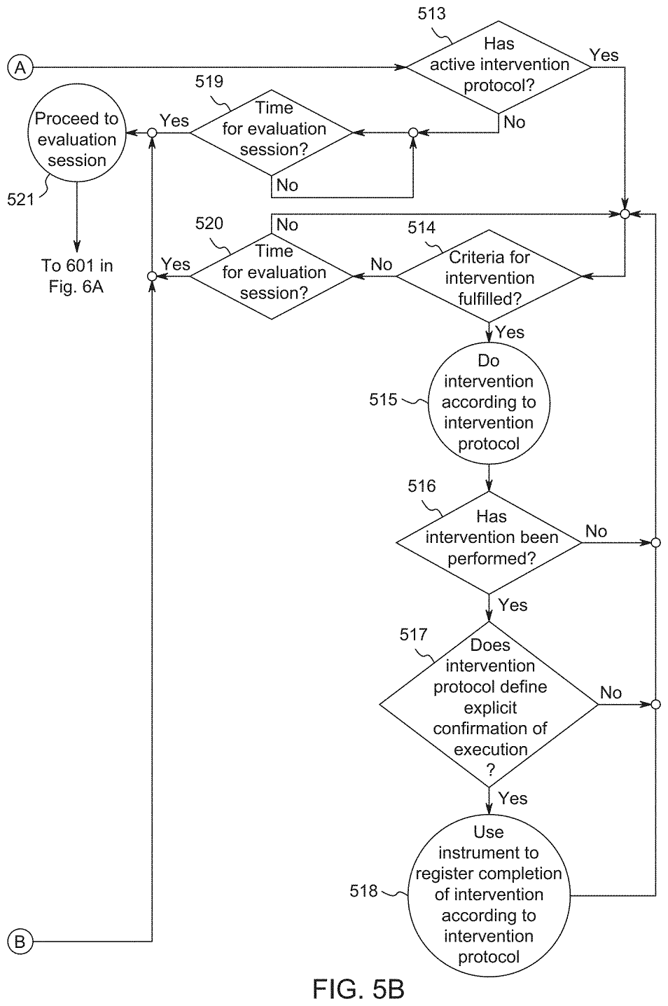

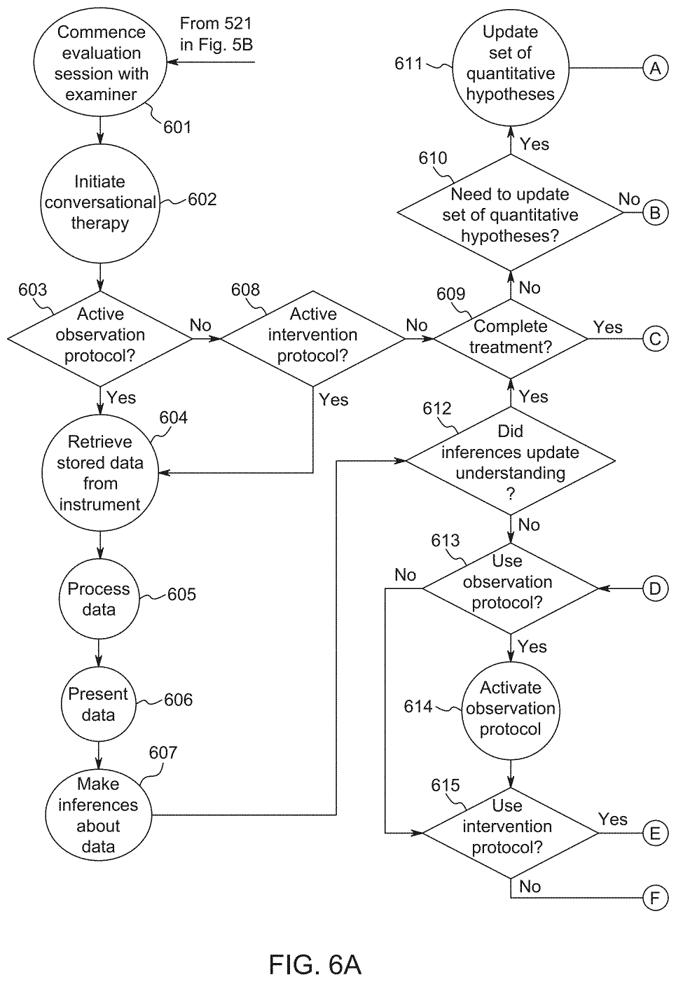

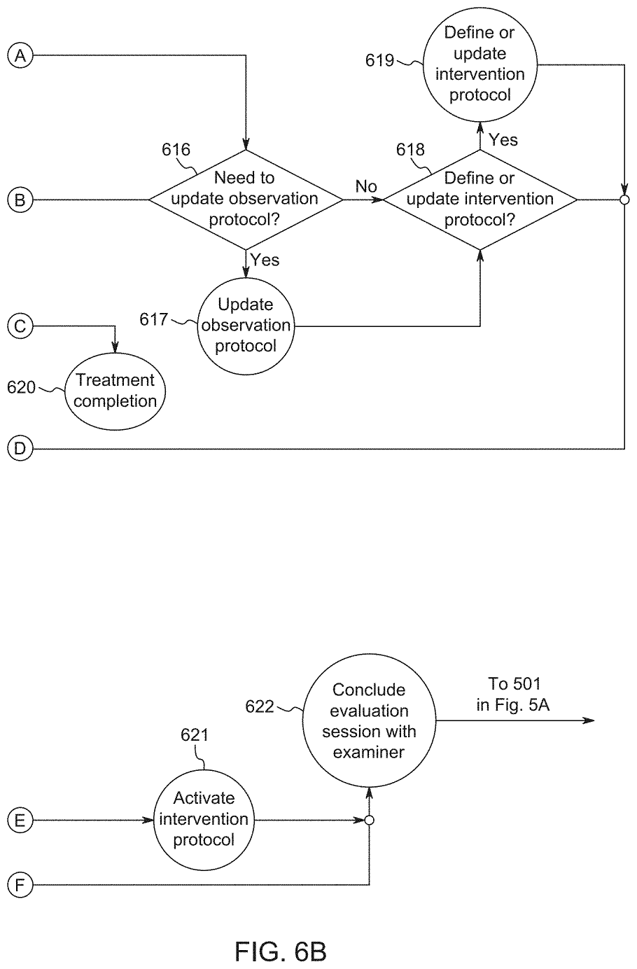

FIGS. 5A-5B show a flow diagram outlining an observation period, according to a specific embodiment of the present disclosure. During the observation period, the individual is typically expected to register observations, i.e. observed occurrences of one or more target phenomena in everyday life situations (block 501). If the individual has an active observation protocol (block 502), i.e. the assessment is associated with an active observation protocol, the individual is typically instructed to be aware of the one or more target phenomena, as defined in the observation protocol (block 503), and upon observation of the target phenomenon (block 504), the individual is typically expected to interact with the portable data acquisition system for registering observations, according to the observation protocol (block 505).

At block 505, the process executed by the data acquisition system generally includes waiting for an interaction, initiating interaction handling, handling an interaction in progress, completing an interaction, persisting an acquired interaction data package, and repeating each step of the process as needed. In some embodiments, the process according to block 505 may be achieved via a user-worn device (e.g., a device worn on the wrist of a user), the user-worn device having a button that receives inputs from a user and transmits one or more signals to a processor, which executes various processes, and optionally, a haptic feedback motor that is operable via inputs received from the processor. More specifically, the user-worn device may initiate interaction handling by acquiring a current time, initiating a timer, and optionally activating the haptic feedback motor. The timer may run and if the haptic feedback motor is activated, a particular haptic pattern may be run by the haptic feedback motor (e.g., a microcontroller unit may control current to the haptic feedback motor to cause a particular vibration pattern—such as on/off for particular periods of time, a particular number of times, and/or at a particular intensity). After a period of time, the haptic feedback motor may be deactivated and the timer may be stopped. A duration of time that has elapsed between the timer start and timer stop may be calculated. The calculated duration of time may be stored in a data storage device, along with the time that the timer was run (e.g., the current time that was acquired). In other embodiments, the process according to block 505 may be achieved by a user-worn device that includes a force sensitive sensor that senses an amount of force applied by a user to a button or the like. For example, the force sensitive sensor may be a force sensitive resistor that changes its resistive value (e.g., in Ohms) depending on how much force is applied thereto by the user. The resistive value can be translated into a parameter that is stored and used by the data acquisition system to influence feedback (e.g., haptic feedback) to denote a magnitude. In another example, the force sensitive sensor may be a force-sensing capacitor or a touch sensor (e.g., a capacitive touch sensor analogous to those used in smartphones and smart watches). Similar to the embodiment above, the user-worn device may also include a processor that executes various processes and optionally a haptic feedback motor that is operable via inputs received from the processor. Similar to the embodiment noted above, the user-worn device may initiate interaction handling by acquiring a current time, initiating a timer, and optionally activating the haptic feedback motor. The timer may run and if the haptic feedback motor is activated, a particular haptic pattern may be run by the haptic feedback motor, which may be based on the amount of force that is sensed (e.g., a stronger sensed force may result in a relatively stronger feedback pattern being emitted and a weaker force may result in a relatively weaker feedback pattern being emitted). Other feedback patterns could be emitted based on a particular mapping between the amount of force sensed and the feedback provided, possibly also involving time as a parameter, such as feedback provided for a maximum duration independently of the force applied. After a period of time, the haptic feedback motor may be deactivated and the timer may be stopped. A duration of time that has elapsed between the timer start and timer stop may be calculated. The calculated duration of time may be stored in a data storage device, along with the time that the timer was run (e.g., the current time that was acquired).

If the individual has an active intervention protocol (block 506), i.e. the assessment is associated with an active intervention protocol, and the criteria for the intervention is fulfilled (block 507) the individual is also expected to carry out an intervention, as defined in the intervention protocol (block 508). Again, similar to what was described for the initiation session, the presently explained method does not necessarily rely on the use of an intervention protocol.

According to block 508, given an active intervention protocol:
1. Determine if intervention criteria have been fulfilled? (blocks 507 and 514)
2. Do intervention according to intervention protocol (blocks 508-509)
   a. Initiate intervention (initiated either by the user or the data acquisition system)
   b. Execute intervention
   c. Complete intervention
   d. (Optional) Persist intervention execution record in a data package (when the system is part of executing the intervention)
3. (Optional) Handle user intervention execution confirmation (blocks 510-511 and blocks 517-518)
   a. Persist intervention confirmation observation

Examples of steps 2-3:
A. User driven:
   a. Initiate intervention: The user realizes that it is time to perform an intervention
   b. Execute intervention: User looks around and counts the colors of things in their vicinity
   c. Complete intervention: When the user feels like resuming normal activity they stop counting colors
   d. Persist intervention execution record in a data package: In this case nothing happens in this step.
   e. Confirmation: The user confirms performing the intervention by pressing the button three times the records of which are stored as interaction data packages
B. System driven:
   a. Initiate intervention: The user has made observations of a target phenomenon and the incidence during the past hour has risen above a set threshold, why the data acquisition/intervention system initiates an intervention to be performed
   b. Execute intervention: The system activates the vibration of the haptic feedback mechanism in a pulsating pattern of 0.1 Hz for 90 seconds
   c. Complete intervention: The system turns off the haptic feedback mechanism
   d. Persist intervention execution record in a data package with:
      i. time stamp of the intervention
      ii. duration of the intervention
      iii. information about the type of intervention
   e. Confirmation: The user confirms that the intervention has been performed by pressing the button three times the records of which are stored as interaction data packages
C. Hybrid:
   a. Initiate intervention:
      i. System: Plays vibration pattern designated for intervention initiation using the haptic feedback mechanism
      ii. User: Presses the button 3 times to signal medium level of stress
      iii. System: Sets intervention parameters (duration of the intervention) based on user input
   b. Execute intervention:
      i. System: Executes vibrations every 3 seconds for 60 seconds
      ii. User: Performs breathing exercise as per the instructions in the intervention protocol while the vibration is felt
   c. Complete intervention: System ceases playing the vibration pattern
   d. Persist intervention execution record in a data package: System persists an intervention data package with:
      i. time stamp of the intervention
      ii. duration of the intervention
      iii. information about the type of intervention
      iv. the user input of stress level (as a parameter for the intervention)
   e. Confirmation: The user confirms that the intervention has been performed by pressing the button three times the records of which are stored as interaction data packages

Depending on whether the intervention has been successfully performed (block 509), and whether the intervention protocol comprises instructions for confirmation of a completed intervention (block 510), the individual may either be instructed to interact with the portable data acquisition system for registering the completion of an intervention (block 511), or continuing with the daily tasks, while being aware of the target phenomena (block 503), as defined in the observation protocol. Similarly, if the assessment does not, at that specific time point have an active observation protocol (block 502), the assessment may still have an active intervention protocol (block 513). In that case, and if the criteria for an intervention are fulfilled (block 514), the individual may be expected to carry out an intervention, as defined in the intervention protocol (block 515). Depending on whether the intervention has been successfully performed (block 516), and whether the intervention protocol comprises instructions for confirmation of a completed intervention (block 517), the individual may be either instructed to interact with the portable data acquisition system for registering the completion of an intervention (block 518), or continuing with the daily tasks, while being aware if the criteria for intervention is fulfilled (block 514). The observation period may run for a time period which may have a predetermined length or wherein the length is determined by specific criteria, such as until a number of observations have been made, or until the individual or the examiner wants to discontinue the observation period for any reason. When the observation period is concluded (blocks 512, 519, 520), the assessment typically progresses to the evaluation session (block 521).

FIGS. 6A-6B show a flow diagram outlining an evaluation session, according to a specific embodiment of the present disclosure. The evaluation session is initiated (block 601) and may comprise conversation between the individual and the examiner (block 602), such as conversational therapy. The individual is typically asked to provide a recollection of the observation period which may be a subjective recollection of the observed occurrences of the target phenomenon, by the individual. If the individual had, during the observation period, an active observation protocol (block 603) and/or an active intervention protocol (block 608), in the cases where the assessment comprises the use of an intervention protocol, the stored data related to observations may be acquired, for example by wireless communication component between the portable data acquisition system and a computer or a tablet in the examiner's office (block 604). In some cases, the examiner may have to be given consent to access the data, by the individual. Following acquisition of the data, it is typically processed (block 605), for example such that the data can be presented (block 606), preferably in a way that comprises graphical overviews and comparisons.

The data may be processed according to block 605 such that the assessment system uses translation of interactions to observations to turn interaction sequences into observations that may be used for assessment of quantitative hypotheses and/or visualizations or other presentations of the data. As prerequisites, there is a time ordered list of interactions and an observation protocol. The steps for processing the data may be as follows:
1) Perform interaction sequencing based on the interaction encoding specified in the observation protocol to obtain next observation
2) (Optional) assign a unique ID to the observation
3) (Optional) enrich the observation with aggregated data from interaction sequences
4) (Optional) enrich the observation with contextual data
5) Assign the phenomenon descriptor to the observation based on the information obtained in step 1-4.
6) While more interactions are available in the interactions time series repeat steps 1-5.

The output of such steps is a time-ordered list of observations. Five example embodiments of step 1 above with different observation protocols include:
1. A “single interaction” observation protocol where each individual interaction is transformed one-to-one to an observation and labeled with the single target phenomenon defined in the observation protocol
2. A “short long interaction” observation protocol where each interaction is transformed one-to-one to an observation and labeled with target phenomena A or B, respectively, depending on whether the duration of the interaction is above or below the time duration threshold defined in the observation protocol
3. A “multi interaction” observation protocol where sequences of interactions of differing length are transformed into observations and labeled with the relevant target phenomenon according to the number of interactions in the sequence, where an interaction belongs to a sequence if the temporal distance to the previous interaction is below the threshold defined in the observation protocol.
4. A “duration to magnitude” observation protocol where each individual interaction is transformed one-to-one to an observation, labeled with the single target phenomenon defined in the observation protocol, and the duration of the interaction is translated to the magnitude of the occurrence of the target phenomenon
5. A “sequence length to magnitude” observation protocol where sequences of interactions of differing length are transformed into a magnitude (e.g. 1-5) and labeled with said magnitude and the target phenomenon according to the observation protocol, where an interaction belongs to a sequence if the temporal distance to the previous interaction is below the threshold defined in the observation protocol.

The presentation of the data is preferably done such that one or more quantitative comparisons between any of the stored data, the quantitative hypotheses, and the recollection can be carried out, for example by the examiner. Based on the quantitative comparisons, the examiner and/or the individual may make inferences about the data (block 607) which may provide the individual and/or the examiner with an understanding, such as an updated understanding, of the suspected condition (block 612).

Making inferences about the data according to block 607 may include assessment of the quantitative hypothesis based on observations and contextual data, as well as additional information provided by the user of the data acquisition system. In the assessment of a quantitative hypothesis the quantitative hypothesis is brought together with relevant observations times series to algorithmically determine the correlation between the observed data and the quantitative hypothesis in question. The procedure includes the following steps:
1. Segment target phenomenon time series according to time period of interest as specified in the quantitative hypothesis
2. Segment source phenomena time series according to time period of interest
3. Apply the inclusion criteria for the target phenomenon specified in the quantitative hypothesis to get a subset of occurrences
4. Apply the inclusion criteria for the source phenomena specified in the quantitative hypothesis to get a subset of occurrences
5. Evaluate the quantitative relation specified in the quantitative hypothesis to calculate the degree of correlation
6. (Optional) Evaluate the degree of correlation according to the stated expectation (prior)

Based on the understanding of the suspected condition of the individual, a continued progress of the assessment of the individual may be decided. The continued progress of the assessment of the individual may typically comprise any, or a combination, of completion of assessment; continuing assessment with interventions; and updating any of i. the quantitative hypotheses, ii. the observation protocol and/or iii. the intervention protocol, and repeating any of steps i-iii, at least partly. If an updated understanding has been established and if the completion of treatment (block 609) is desired, the assessment may be completed (block 620). If the completion of the assessment is not desired (block 609), the assessment may, if desired (block 610) be continued with an updated set of quantitative hypotheses (block 611) (which may use a process similar to that described hereinabove with respect to block 404 in FIG. 4). Furthermore, the observation protocol may, if desired (block 616), be updated (block 617) (e.g., according to the various processes described herein with respect to block 406), and additionally the intervention protocol may, if desired (block 618), be updated (block 619) (which may be completed according to the processes of block 410 (FIG. 4)). Following updating of said hypotheses and protocols, or if a sufficiently updated understanding has not been established (block 612), the observation protocol may be considered for activation (block 613). If activation is desired, the observation protocol is activated (block 614). Similarly, the intervention protocol, in instances where the method uses the optional intervention protocol, may be considered for activation (block 615), and if desired, it may be activated (block 621). Thereafter, the evaluation session is concluded (block 622), and any parts of the initiation session, the observation period, and/or the evaluation session may be repeated, at least partly. For example, the individual may be referred back to the observation period, wherein the individual is to experience everyday life situations (block 501) and may make observations according to the observation protocol and, if present, use the intervention protocol.

In a combined example with definition of observation and intervention protocol:

Definition of the Observation Protocol:
1. Target phenomenon: Arousal experienced by the individual as a change in breathing patterns
2. Interaction pattern encoding: Press the button once when the target phenomenon is experienced
3. Provide human-readable instructions for the selected encoding pattern for the individual
4. Remove the “single press” encoding from the set of available encodings
5. Persist the target phenomenon definition

Definition of the Intervention Protocol:
1. Intervention: Count colors of things in the immediate vicinity of the individual
2. Trigger criteria: An observation of arousal has been made
3. Intervention execution: The individual performs the intervention immediately after making the observation of arousal
4. Intervention completion:
   a. When the individual feels sufficiently downregulated they stop counting colors of things in their vicinity
   b. The individual is to press the button twice (less than 800 ms apart) to record a confirmation of the intervention execution

Further aspects of the invention are provided by the subject matter of the following clauses:

In an aspect, a method for assessment of one or more suspected conditions of an individual by an examiner, the method comprising: one or more initiation sessions, involving conversation, comprising: collaborative identification, between the individual and the examiner, of one or more features of the suspected condition, and one or more subject-specific phenomena of presentation, selection of one or more target phenomena, of said subject-specific phenomena of presentation, generation of a number of quantitative hypotheses, definition of an observation protocol, comprising or consisting of instructions for how the individual is to self-monitor and register observations in the form of stored data, by interaction with a portable data acquisition system, upon each occurrence of the target phenomenon is observed by the individual; one or more observation periods, wherein the individual is instructed to carry out the observation protocol upon observation of the target phenomenon, thereby self-monitoring and registering observations on the portable data acquisition system; and one or more evaluation sessions, involving conversation, comprising; acquisition of the stored data, such as the observation data, on the portable data acquisition system; recollection of the observation period, by the individual; one or more quantitative comparisons between any of: the stored data; the quantitative hypotheses; and the recollection; inferring, based on the quantitative comparison, an understanding of the suspected condition of the individual; and concluding, based on the understanding of the suspected condition of the individual, a continued progress of the assessment of the individual.

The method according to any one of the preceding aspects, wherein the suspected condition comprises one or more mental conditions, and/or one or more physiological conditions.

The method according to any one of the preceding aspects, wherein the features of the suspected condition comprises or consists of one or more physiological features, and/or one or more mental health related features.

The method according to any one of the preceding aspects, wherein the subject-specific phenomena of presentation are how the individual consciously experiences features of the suspected condition, such as bodily experienced, environmentally experienced, and/or mentally experienced.

The method according to any one of the preceding aspects, wherein the subject-specific phenomena of presentation are one or more of: bodily experienced precursors to a mental state, such as a somatic marker or mental awareness; environmentally experienced; mentally experienced, such as conscious awareness of a phenomenon, to one or more of the features of the suspected condition, for example: awareness of mental states, such as awareness of specific cognitions, such as psychotic perceptions, ruminative thought patterns, attention towards threats, depressive cognitions; or bodily sensations, such as pain.

The method according to any one of the preceding aspects, wherein the target phenomenon of said subject-specific phenomena of presentation, comprises or consists of any of the subject-specific phenomenon of presentation that: has the biggest impact on the individual's life; is bothering the individual the most; is the most directly noticeable in everyday situations of the individual; is considered to be the most relevant indicator for treatment or in relation to a diagnosis; is considered to be of greatest clinical interest and/or relevance; is believed to lead to the greatest reduction of uncertainty about the suspected condition; and/or is relevant for the suspected condition.

The method according to any one of the preceding aspects, wherein the preparatory session comprises or consists of conversational therapy, for example therapy according to, at least a part of, the conversational model.

The method according to any one of the preceding aspects, wherein the preparatory session comprises or consists of conversation between the individual and the examiner, and wherein the examiner is a conversational software agent.

The method according to any one of the preceding aspects, wherein the observation protocol comprises instructions for self-monitoring and registering of observations by interaction with the portable data acquisition system in a way that is unique for each target phenomenon, such that each target phenomenon is associated with a unique type of interaction.

The method according to any one of the preceding aspects, wherein each quantitative hypothesis comprises or consists of an expectation about the occurrences of the target phenomena.

The method according to any one of the preceding aspects, wherein the set of the quantitative hypotheses are generated by the individual and/or the examiner.

The method according to any one of the preceding aspects, wherein the set of the quantitative hypothesis comprises or consists of a number of hypotheses of the quantitative distribution of the occurrences of the target phenomenon.

The method according to any one of the preceding aspects, wherein the set of quantitative hypotheses comprises or consists of one or more hypotheses concerning the distribution of occurrences of the target phenomenon, in terms of any of: time, such as specific time intervals during the day; situations, such as when the individual performs specific actions; events, such as when specific events occur to the individual; physical locations, such as when the individual is located at specific physical locations; presence of living beings, such as when certain people or animals are near and/or interacting with the individual.

The method according to any one of the preceding aspects, wherein the examiner and/or the individual registers the quantitative hypotheses by interaction with an interactive system, such as a computational device.

The method according to any one of the preceding aspects, wherein the individual during the observation period, carries out normal daily tasks, such that the stored data comprises or consists of observations made in everyday life situations.

The method according to any one of the preceding aspects, wherein the individual further registers the observed magnitude of the occurrence of the target phenomenon by interacting with the portable data acquisition system, and/or repeatedly interacting with the portable data acquisition system, such as for a duration of time related to the magnitude of the target phenomenon.

The method according to any one of the preceding aspects, wherein the individual further registers the duration of the observed occurrence of the target phenomenon by interacting with the portable data acquisition system, and/or repeatedly interacting with the portable data acquisition system, such as for a duration of time related to the duration of the target phenomenon.

The method according to any one of the preceding aspects, wherein the observation period is carried out for a time period of at least 3 days, such as between 3 days and 90 days.

The method according to any one of the preceding aspects, wherein the evaluation session comprises or consists of conversational therapy, for example therapy according to, at least a part of, cognitive behavioral therapy (CBT).

The method according to any one of the preceding aspects, wherein the preparatory session comprises or consists of conversation between the individual and the examiner, and wherein the examiner is a conversational software agent.

The method according to any one of the preceding aspects, wherein the recollection of the observation period is a subjective recollection of the observed occurrences of the target phenomenon, by the individual.

The method according to any one of the preceding aspects, wherein the recollection of the observation period, comprises frequency and time distribution of the observed occurrences of the target phenomenon.

The method according to any one of the preceding aspects, wherein the recollection further comprises a mapping of the events and/or factors that potentially lead to the triggering of the target phenomenon.

The method according to any one of the preceding aspects, wherein, prior to acquisition of the stored data, consent to acquire the stored data is obtained from the individual, such as by the examiner.

The method according to any one of the preceding aspects, wherein acquisition of the stored data requires an access key, such as a one-time use alphanumeric key, provided by the individual.

The method according to any one of the preceding aspects, wherein a visual overview of the observations is generated, such as a timeline and/or a heat map of the observations, such that patterns in the data can be inferred.

The method according to any one of the preceding aspects, wherein the quantitative comparisons comprise a visual overview of any of the stored data, the recollection, and/or the quantitative hypotheses.

The method according to any one of the preceding aspects, wherein location data and/or time data, such as time-stamps, are used to bin the observations to a number of places.

The method according to any one of the preceding aspects, wherein the places have pseudonymized or randomized labels, such as A, B and C, or wherein the places are physical locations, such as a workplace, a home and during travel between two physical locations, such as commute between home and workplace.

The method according to any one of the preceding aspects, wherein the continued progress of the assessment of the individual comprises any, or a combination, of: completion of assessment; continuing assessment with interventions; and updating any of i. the quantitative hypotheses, ii. the observation protocol and/or iii. the intervention protocol, and repeating any of steps i-iii, at least partly.

The method according to any one of the preceding aspects, wherein the initiation session further comprises definition of an intervention protocol, comprising or consisting of instructions of how the individual is to perform one or more self-administered interventions.

The method according to any one of the preceding aspects, wherein the intervention protocol further comprises definitions and/or instructions of when the individual is to perform the self-administered intervention, such as upon occurrence of the target phenomenon is observed by the individual or upon provision of an intervention prompt by the portable data acquisition system.

The method according to any one of the preceding aspects, wherein the portable data acquisition system is configured to provide the intervention prompt at any of: after a predetermined number of registered observations, at regular time intervals, at random time intervals, at specific time points during the day, below, at, and/or above a specific frequency of observations, at certain locations and/or after observations with an magnitude below, at, and/or above a predetermined threshold.

The method according to any one of the preceding aspects, wherein the intervention protocol further comprises instructions of how the individual is to register a completion of the intervention, upon each completion of the intervention.

The method according to any one of the preceding aspects, wherein the portable data acquisition system comprises a number of input component, for interaction with the portable data acquisition system, such as one or more buttons, a number of pressure sensors for registering squeezing of the portable data acquisition system, a touch screen, a fingerprint sensor, and/or a microphone.

The method according to any one of the preceding aspects, wherein the portable data acquisition system is configured for multiple different types of interactions, by a single or by multiple input component.

The method according to any one of the preceding aspects, wherein the portable data acquisition system is configured for multiple different types of interactions, and wherein each target phenomenon and/or, the completion of an intervention, is associated with a unique type of interaction.

The method according to any one of the preceding aspects, wherein the portable data acquisition system, upon interaction, automatically stores data, such that each interaction is associated with unique interaction data.

The method according to any one of the preceding aspects, wherein the interaction data comprises contextual information, such as selected from the list including: force data, such as the force by which the button is pressed, time data, such as one or more time-stamps, location data, temperature data, orientation data, such as absolute orientation of the device, audio recording, such as audio features, present Bluetooth devices, present WiFi devices/hotspots, devices using wireless communication protocols and/or their signal strength (e.g. Bluetooth or WiFi) present in the vicinity of the portable data acquisition system, intensity and other features of the ambient light.

The method according to any one of the preceding aspects, wherein the interaction data comprises information related to physiological measurements of the individual, such as heart rate and heart rate variability (HRV) measurements, galvanic skin response (GSR) measurements, motion sensors, and/or temperature measurements.

The method according to any one of the preceding aspects, wherein the observation information is stored locally on the portable data acquisition system, or on a networked server, or a combination thereof.

The method according to any one of the preceding aspects, wherein the portable data acquisition system is configured to provide feedback upon interaction, such as haptic feedback, auditory feedback and/or visual feedback.

The method according to any one of the preceding aspects, wherein the portable data acquisition system is wearable, such as worn on the body or attached to or embedded in clothes, such as wherein the data acquisition device comprises a wristband, a lanyard, and/or a belt mount.

The method according to any one of the preceding aspects, wherein the portable data acquisition system comprises a power source, a memory and a processing unit.

The method according to any one of the preceding aspects, wherein the portable data acquisition system is reconfigurable and comprises an input component, for programming and/or reprogramming instructions stored on the memory.

A portable data acquisition system for use in assessment of one or more suspected conditions of an individual by an examiner, said portable data acquisition system being configured for storing data related to observations of occurrences of target phenomena, upon being provided an interaction by an individual.

The portable data acquisition system according to any one of the preceding aspects, wherein the portable data acquisition system comprises one or more input component, for interaction with the portable data acquisition system, such as one or more buttons, a number of pressure sensors for registering squeezing of the portable data acquisition system, a touch screen, a fingerprint sensor, a camera, and/or a microphone.

The portable data acquisition system according to any one of the preceding aspects, wherein the portable data acquisition system is configured for multiple different types of interactions, by a single or by multiple input component.

The portable data acquisition system according to any one of the preceding aspects, wherein the portable data acquisition system is configured for multiple different types of interactions, and wherein each target phenomenon and/or, the completion of an intervention, is associated with a unique type of interaction.

The portable data acquisition system according to any one of the preceding aspects, wherein the portable data acquisition system is configured to, upon being provided an interaction, automatically store data, such that each interaction is associated with unique interaction data.

The portable data acquisition system according to any one of the preceding aspects, wherein the interaction data comprises contextual information, such as selected from the list including: force data, such as the force by which the button is pressed, time data, such as one or more time-stamps, location data, temperature data, orientation data, such as absolute orientation of the device, audio recording, such as audio features, present Bluetooth devices, present WiFi devices/hotspots, devices using wireless communication protocols and/or their signal strength (e.g. Bluetooth or WiFi) present in the vicinity of the portable data acquisition system, intensity and other features of the ambient light.

The portable data acquisition system according to any one of the preceding aspects, wherein the interaction data comprises information related to physiological measurements of the individual, such as heart rate and heart rate variability (HRV) measurements, galvanic skin response (GSR) measurements, motion sensors, and/or temperature measurements.

The portable data acquisition system according to any one of the preceding aspects, wherein the portable data acquisition system is configured to store data locally, or on a networked server, or a combination thereof.

The portable data acquisition system according to any one of the preceding aspects, wherein the portable data acquisition system is configured to provide feedback upon interaction, such as haptic feedback, auditory feedback and/or visual feedback.

The portable data acquisition system according to any one of the preceding aspects, wherein the portable data acquisition system is wearable, such as worn on the body or attached to or embedded in clothes, such as wherein the data acquisition device comprises a wristband, a lanyard, and/or a belt mount.

The portable data acquisition system according to any one of the preceding aspects, wherein the portable data acquisition system comprises a power source, a memory and a processing unit.

The portable data acquisition system according to any one of the preceding aspects, wherein the portable data acquisition system is reconfigurable and comprises an input component, for programming and/or reprogramming instructions stored on the memory.

The portable data acquisition system according to any one of the preceding aspects, wherein said portable data acquisition system is configured for being interacted with upon each occurrence of a target phenomenon is observed by an individual, according to the method of any one of the preceding aspects.

What is claimed is:

1. A method for assessment of one or more suspected conditions of an individual, the method comprising:
receiving, by an analysis component, collaborative identification data pertaining to one or more features of a suspected condition and one or more subject-specific phenomena of presentation;
selecting, by the analysis component, one or more target phenomena from the one or more subject-specific phenomena of presentation;
receiving, by the analysis component, one or more quantitative hypotheses for the one or more target phenomena;
defining, by the analysis component, an observation protocol comprising instructions for how the individual is to self-monitor and register observations in the form of stored data, by interaction with a portable data acquisition system communicatively coupled to the analysis component, upon each occurrence of the target phenomena is observed by the individual;
directing, by the portable data acquisition system, the individual to provide the stored data to the portable data acquisition system in accordance with the observation protocol upon observation of a target phenomenon of the one or more target phenomena during one or more observation periods;
transmitting the stored data from the portable data acquisition system to the analysis component;
directing the individual to recall each observation period;
completing, by the analysis component, one or more quantitative comparisons between any of:
the stored data;
the one or more quantitative hypotheses; and
the recollection;
inferring, based on the quantitative comparison, the one or more suspected conditions of the individual; and
determining, based on the inferred one or more suspected conditions of the individual, a continued progress of the assessment of the individual.

2. The method of claim 1, wherein the suspected condition comprises at least one of:
one or more mental conditions; and
one or more physiological conditions.

3. The method of claim 1, wherein the one or more subject-specific phenomena of presentation are how the individual consciously experiences features of the suspected condition.

4. The method of claim 1, wherein further comprising conducting a conversational therapy session with the individual.

5. The method of claim 4, wherein the conversational therapy session comprises a conversation between the individual and a conversational software agent of the analysis component or the portable data acquisition component.

6. The method of claim 1, wherein the observation protocol comprises instructions for self-monitoring and registering of observations by interaction with the portable data acquisition system in a way that is unique for each target phenomenon such that each target phenomenon is associated with a unique type of interaction with the portable data acquisition system.

7. The method of claim 1, wherein each one of the one or more quantitative hypotheses comprises an expectation about the occurrences of the target phenomena.

8. The method of claim 1, wherein the one or more quantitative hypotheses comprises a number of hypotheses of the quantitative distribution of the occurrences of the target phenomena.

9. The method of claim 1, wherein the individual further registers the observed magnitude of the occurrence of the target phenomenon by interacting with the portable data acquisition system for a duration of time related to the magnitude of the target phenomena.

10. The method of claim 1, wherein each observation period is carried out for a time period of at least 3 days.

11. The method of claim 1, wherein directing the individual to recall comprises directing the individual to complete a subjective recollection of the observed occurrences of the target phenomenon.

12. The method of claim 1, wherein obtaining the stored data comprises providing an access key to authenticate with the portable data acquisition system.

13. The method of claim 1, further comprising direction the individual to carry out an intervention protocol.

14. A system for assessment of one or more suspected conditions of an individual, the system comprising:
an assessment device;
a portable data acquisition system communicatively coupled to the assessment device; and
an external device coupled to the assessment device,
wherein the assessment device is configured to:
receive collaborative identification data pertaining to one or more features of a suspected condition and one or more subject-specific phenomena of presentation,
receive one or more quantitative hypotheses for the one or more target phenomena, and
define an observation protocol comprising instructions for how the individual is to self-monitor and register observations;
wherein the portable data acquisition system is configured to receive the registered observations from the individual in accordance with the observation protocol upon observation of a target phenomenon of the one or more target phenomena during one or more observation periods,
wherein the assessment device is further configured to:
obtain the data from the portable data acquisition system,
direct the individual to recall each observation period,
complete one or more quantitative comparisons between any of the stored data, the quantitative hypotheses, and the recollection,
infer, based on the quantitative comparison, the one or more suspected conditions of the individual, and
determine, based on the inferred one or more conditions of the individual, a continued progress of the assessment of the individual, and
wherein the external device receives a data file from the assessment device, the data file containing the continued progress of the assessment of the individual and presents the data file to a user.

15. The system of claim 14, wherein the portable data acquisition system comprises a plurality of user interface components for interaction with the portable data acquisition system, as the plurality of user interface components selected from one or more buttons, a number of pressure sensors for registering squeezing of the portable data acquisition system, a touch screen, a fingerprint sensor, and a microphone.

16. The system of claim 14, wherein the portable data acquisition system is configured for multiple different types of interactions, by a single or by multiple input means.

17. The system of claim 14, wherein the portable data acquisition system is configured for multiple different types of interactions, and wherein each target phenomenon is associated with a unique type of interaction.

18. The system of claim 14, wherein the portable data acquisition system, upon interaction, automatically stores data, such that each interaction is associated with unique interaction data.

19. The system of claim 14, wherein the interaction data comprises contextual information, such as selected from the list including: force data, time data, location data, temperature data, orientation data, audio recording, present Bluetooth devices, present WiFi devices, devices using wireless communication protocols, a signal strength present in the vicinity of the portable data acquisition system, and intensity of the ambient light.

20. The system of claim 14, wherein the interaction data comprises information related to physiological measurements of the individual, such as heart rate and heart rate variability (HRV) measurements, galvanic skin response (GSR) measurements, motion sensors, and/or temperature measurements.
